(19) United States
(12) Patent Application Publication
(10) Pub. No.: US 2021/0315837 A1
(43) Pub. Date: Oct. 14, 2021

(54) CANNABINOIDS FOR THE TREATMENT OF GRAM-POSITIVE INFECTIONS INCLUDING ANTIBIOTIC-RESISTANT BACTERIAL STRAINS

(71) Applicants: Nemus Bioscience, Inc., Long Beach, CA (US); University of Mississippi, University, MS (US)

(72) Inventors: Brian Murphy, Newport Coast, CA (US); Mahmoud El Sohly, Oxford, MS (US); Waseem Gul, Oxford, MS (US); Melissa Jacob, Burnet, TX (US)

(21) Appl. No.: 17/273,745

(22) PCT Filed: Sep. 5, 2019
(86) PCT No.: PCT/US19/49671

§ 371 Date: Mar. 5, 2021

Related U.S. Application Data
(60) Provisional application No. 62/727,460, filed on Sep. 5, 2018.

(51) Int. Cl. A61K 31/05; A61K 45/06; A61P 31/04; A61K 31/352

(52) U.S. Cl. CPC A61K 31/05; A61K 31/352; A61P 31/04; A61K 45/06

(57) ABSTRACT

The present invention provides compositions and methods for treating or preventing a gram-positive bacterial infection. In one aspect, the composition comprises at least two cannabinoids.

### CROSS REFERENCE TO RELATED APPIJCATIONS

This application claims priority to U.S. Provisional Application No. 62/727,460, filed Sep. 5, 2018 which is hereby incorporated by reference herein in its entirety.

### BACKGROUND OF THE INVENTION

As resistance to antibiotics becomes more common there is greater need for alternative treatments. There remains a powerful need for particular agents that are effective as bacteriostatic versus bactericidal agents against a wide range of gram positive bacteria, including but not limited to: Clostridium difficile, Carbapenem-resistant Enterobacteriaceae (CRE), drug-resistant Neisseria gonorrhoeae (microorganisms with a threat level of urgent); Multidrug-resistant Acinetobacter, drug-resistant Campylobacter, ESBL-producing Enterobacteriaceae, Vancomycin-resistant Enterococcus (VRE), multidrug-resistant Pseudomonas aeruginosa, drug-resistant non-typhoidal Salmonella, drug-resistant Salmonella typhimurium, drug-resistant Shigella, MRSA, drug-resistant Streptococcus pneumoniae, drug-resistant tuberculosis (microorganisms with a threat level of serious); and Vancomycin-resistant Staphylococcus aureus (VRSA), Erythromycin-resistant Group A Streptococcus, and Clindamycin-resistant Group B Streptococcus (microorganisms with a threat level of concerning), and additionally other bacteria such as Propionibacterium acnes (the bacteria that causes acne and which is displaying resistance to a battery of antibiotics) and mupirocin-resistant MRSA (Mup-MRSA). The present invention addresses this unmet need.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts experimental results demonstrating that the combination of CBD and CBG showed synergy against Mupirocin-resistant MRSA.

FIG. 2 depicts experimental results demonstrating combinations showed improved effect over monotherapy.

FIG. 3 depicts experimental results of triple therapy compared to monotherapy.

FIG. 4 depicts experimental results demonstrating the combination of CBD and CBG is synergistic.

FIG. 5 depicts experimental results demonstrating e combination of CBD and CBN is synergistic.

FIG. 6 depicts the experimental results of a checkerboard experiment demonstrating the optimum combination. The optical density at 530 nM was determined after 23 hours of incubation. The optimum combination is denoted by the circle.

FIG. 7 depicts the results of a time to kill experiment comparing the combination of CBD (1.25 μg/ml)+CBG (1.25 μg/ml) to CBD (0.5 μg/ml) alone, CBG (0.5 μg/ml) methicillin (50 μg/ml) and DMSO.

FIG. 8 depicts the results of a time to kill experiment comparing the combination of cm) (1.25 μg/mi)+CBG (1.25 μg/ml) to CBD (2.5 μg/ml) alone, CBG (2.5 μg/ml) alone, CBD (1.25 μg/ml) alone, CBG (1.25 μg/ml) alone, methicillin (5 μg/ml) and DMSO.

FIG. 9 depicts a checkerboard experiment wherein after incubation, the wells were pinned to agar to determine the bactericidal potential of all combinations.

FIG. 10 depicts the experimental results of growth inhibition assay comparing the combination of CBD and CBG to CBD alone and CEG alone,

FIG. 11 depicts and image of the growth inhibition assay.

FIG. 12 depicts the results of the growth inhibition test of MRS 33591 using CBD and CBG, alone and in combination.

FIG. 13 depicts the isobologram of CBD/CBG combination against MRS 33591.

FIG. 14 depicts the results of the growth inhibition test of MRS 33591 using CBN and CBG, alone and in combination.

FIG. 15 depicts the isobologram of CBN/CBG combination against MRS 33591.

FIG. 16 depicts the results of the growth inhibition test of MRS 33591 using CBD and CBN, alone and in combination.

FIG. 17 depicts the isobologram of CBD/CBN combination against MRS 33591.

FIG. 18 depicts the results of the growth inhibition test of MRS 1696 using CBD and CBG, alone and in combination.

FIG. 19 depicts the isobologram of CBD/CBG combination against MRS 1696.

FIG. 20 depicts the results of the growth inhibition test of MRS 1696 using CBN and CBG, alone and in combination.

FIG. 21 depicts the isobologram of CION/03G combination against MRS 1696.

FIG. 22 depicts the results of the growth inhibition test of MRS 1696 using CBD and CBN, alone and in combination.

FIG. 23 depicts the isobologram of CBD/CBN combination against MRS 1696.

FIG. 24 depicts the results of the growth inhibition test of MRS 1708 using CBD and CBG, alone and in combination.

FIG. 25 depicts the isobologram of CBD/CBG combination against MRS 1708.

FIG. 26 depicts the results of the growth inhibition test of MRS 1708 using CBN and CBG, alone and in combination.

FIG. 27 depicts the isobologram of CBN/CBG combination against MRS 1708.

FIG. 28 depicts the results of the growth inhibition test of MRS 1708 using CBD and CBN, alone and in combination.

FIG. 29 depicts the isobologram of CBD/CBN combination against MRS 1708.

FIG. 30 depicts the results of the growth inhibition test of MRS 1717 using CBD and CBG, alone and in combination.

FIG. 31 depicts the isobologram of CBD/CBG combination against MRS 1717.

FIG. 32 depicts the results of the growth inhibition test of MRS 1717 using CBN and CBG, alone and in combination.

FIG. 33 depicts the isobologram of CBN/CBG combination against MRS 1717.

FIG. 34 depicts the results of the growth inhibition test of MRS 1717 using CBD and CBN, alone and in combination.

FIG. 35 depicts the isobologram of CBD/CBN combination against MRS 1717.

FIG. 36 depicts a time to kill assay comparing Cannabidiol and Cannabigerol vs. Mupirocin-resistant MRS.

### DETAILED DESCRIPTION

The present invention relates to compositions and methods for treating bacterial infections. In one aspect, the invention provides compositions comprising two or more cannabinoids. For example, the present invention includes compositions comprising two or more of Cannabidiol (CBD) or a CED derivative, Cannabinol (CEN) or a CBN derivative, and Cannabigerol (CBG) or a CBG derivative. The present invention also includes methods of using the compositions for preventing or reducing a bacterial infection and diseases and disorders related to bacterial infections. For example, in one embodiment, the bacterial infection is a gram-positive infection. In one embodiment, the bacterial infection is an antibiotic resistant infection.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generality performed according to conventional methods in the art and various general references (e.g., Sambrook and Russell, 2012, Molecular Cloning, A Laboratory Approach, Cold Spring Harbor Press, Cold Spring Harbor, NY, and Ausubel et al., 2002, Current Protocols in Molecular Biology, John Wiley & Sons, NY), which are provided throughout this document.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±120%, ±10%, ±5%, ±1%, or ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, matte, fumaric, succinic, tartaric, amsonic, pamoic, p-totunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantoth.enic, benzenesulfonic (besylate), stearic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function, Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “cannabinoid” refers to compounds which can be obtained by chemical synthesis, chemical modification, or obtained from plant materials derived front one or more Cannabis plants. Exemplary cannabinoids include, but are not limited to, cannabidiol (cm)), cannabinol (CBN), cannabichromene (CBC), cannabigeroi (CBG), cannabidivarin (CBV), Δ8-Tetrahydrocannabinot (Δ8-THC), and derivatives thereof. The cannabinoids of the present invention may be synthesized using techniques well-known in the art of organic synthesis. The starting materials and intermediates required for the synthesis may be obtained from commercial sources or synthesized according to methods known to those skilled in the art.

The term “Cannabis plant(s)” encompasses wild type Cannabis saliva and also variants thereof, including cannabis chemovars which naturally contain different amounts of the individual cannabinoids, Cannabis saliva subspecies indica including the variants indica and katiristanica, Cannabis indica and also plants which are the result of genetic crosses, self-crosses or hybrids thereof. The term “Cannabis plant material” is to be interpreted accordingly as encompassing plant material derived from one or more cannabis plants. “Cannabis plant material” includes dried cannabis biomass.

As used herein, the term “minimum inhibitory concentration (MIC)” refers to the lowest concentration of an antimicrobial agent that will inhibit the visible growth of a microorganism after overnight incubation. MIC values against bacteria may be determined by standard methods. See also P. A. Wayne, Methods for Dilution Antimicrobial Tests for Bacteria that Grow Aerobically; Approved Standard, Ninth Edition, 2012, CLSI Document M07-A9, Vol. 32 No. 2, which is incorporated by reference herein in its entirety.

As used herein, the term “potency” refers to the dose needed to produce half the maximal response (ED50).

As used herein, the term “efficacy” refers to the maximal effect (Emax) achieved within an assay.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2,7, 3, 4, 5, 5.3, and 6. This applies regardless of the breadth of the range.

### Description

The present invention is based, in part, on the unexpected finding that cannabinoid cocktails have significant synergistic bactericidal activity against Methicillin-resistant Staphylococcus aureus (MRSA) and other gram-positive bacteria. Accordingly, the present invention relates to compositions and methods of treating or preventing a bacterial infection in a subject.

In one aspect, the invention provides compositions comprising two or more cannabinoids. For example, in one embodiment, the composition comprises two or more of Cannabidiol (CBD), a CBD derivative, Cannabinol (CBN), a CBN derivative. Cannabigerol (CBG), and a CBG derivative. In one embodiment, the composition comprises at least two or more of CBD, CBN and CBG. in one embodiment, the composition comprises CRD, CBN and CBG.

The present invention also provides methods for treating gram-positive bacterial infections, such as infections of Staphylococcus aureus, Hemolytic Stept, P. acnes, Clostridium, Bacillus, and Listeria, in a subject. In one embodiment, the method comprises administering to the subject an effective amount of two or more of Cannabidiol (CBD), a CBD derivative, Cannabinol (CBN), a CBN derivative. Cannabigerol (CBG), and a CBG derivative. In one embodiment, the two or more cannabinoids are administered concurrently. In one embodiment, the two or more cannabinoids are administered as a single formulation.

In one aspect, the methods also treat disease or disorder associated with a bacterial infection. For example, exemplary diseases or disorders treated or prevented by the methods of the invention include cellulitis, impetigo, erysipelas, boils, acne, abscesses, epidermolysis bullosa, a bullous disease, toxic epidermal necrolysis, an infection secondary to environmental allergens (or drug reactions, and staphylococcal scalded skin syndrome).

The invention also provides methods of preventing or reducing the growth or proliferation of microorganisms and/or biofilm-embedded microorganisms on at least one surface. For example, in one embodiment, the method comprises providing at least one surface; providing a composition comprising at least two cannabinoids, and applying the composition to the at least one surface in an amount sufficient to prevent or reduce the growth or proliferation of microorganisms or biofilm-embedded microorganisms on the at least one surface.

The methods of the invention can reduce or prevent proliferation of microorganisms and/or biofilm-embedded microorganisms on surfaces such as subject's body, a medical device, a solid surface, glass surface, a metal surface, a paper surface, or a polymer surface.

### Compositions

In some embodiments, the present invention provides compositions for treating a bacterial infection or a disease or disorder related to a bacterial infection in a subject, In one embodiment, the composition of the invention comprises two or more cannabinoids. In one embodiment, the composition comprises Cannabidiol (CBD) or a CBD derivative, and Cannabinol (CBN) or a CBN derivative. in one embodiment, the composition comprises CBD or a CBD derivative and Cannabigerol (CBG) or a CBG derivative. In one embodiment, the composition comprises CBN or a CBN derivative and CBG or a CBG derivative, In one embodiment, the composition comprises CBD or a CBD derivative, CBN or a CBN derivative, and CBG or a CBG derivative.

Exemplary CBG derivatives include, but are not limited to Tetrahydrocannabigerol, 6,7-Epoxycannabigerol, Cannabigerolic acid, Tetrahydrocannabigerolic acid, (Z)-Cannabigerorcin, and Methyl tetrahydrocannabigerol.

Exemplary CBN derivatives include, but are not limited to, 8-Hydroxycannabinol, Cannabinotic acid, and Cannabinol acetate.

Exemplary CBD derivatives include, but are not limited to, Cannabidiolic acid, Monomethoxy cannabidiol, Cannabielsoin, Carmabichromene, and Cannabichromeorcin.

In one embodiment, the composition further comprises Δ8-Tetrahydrocannabinol (Δ8-THC) or a Δ8-THC derivative. Exemplary Δ8-THC derivatives include, but are not limited to, 2-dimethylamino-Δ8 THC, 2,8-dinitro-Δ8 THC, 2-butylamino-Δ8 THC, and Δ8-Tetrahydrocannabinol quinone.

The compositions useful within the invention may further comprise at least one additional antibacterial agent. Non-limiting examples of the at least one additional antibacterial agent are daptomycin (Cubicin), levotloxacin, doxycycline, neomycin, clindamycin, minocycline, gentamycin, rifampin, chlorhexidine, chloroxylenol, methylisothizoione, thymol, α-terpineol, cetylpyridinium chloride, hexachlorophene, triclosan, nitrofurantoin, erythromycin nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linexolid, temafloxacin, tosufloxacin, clinatloxacin, sulbactam, clavulanic acid, amphotericin B, fluconazole, itraconazole, ketoconazole, nystatin, penicillins, cephalosporins, carbepenems, beta-lactams antibiotics, aminoglycosides, macrolides, lincosamides, glycopeptides, tetracylines, chloramphenicol, quinolones, fucidines, sulfonamides, trimethoprims, rifamycins, oxalines, streptogramins, lipopeptides, ketolides, polyenes, azoles, echinocandines, and any combination thereof.

In one embodiment, the two or more cannabinoids act synergistically in preventing, reducing or disrupting a bacterial infection. In one embodiment, the two or more cannabinoids and the at least one additional agent act synergistically in preventing, reducing or disrupting a bacterial infection. A synergistic effect may be calculated, for example, using suitable methods such as, for example, the Sigmoid-Ema, equation (Holford & Scheiner, 19981, Clin. Pharmacokinet. 6: 429-453), the equation of Loewe additivity (Loewe & Muischnek, 1926, Arch. Exp. Pathol Phannacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Methods

In some embodiments, the invention provides methods of treating a bacterial infection or a disease or disorder associated with a bacterial infection, In one embodiment, the method comprises administering to the subject an effective amount of at least two cannabinoids. In one embodiment, the bacterial infection is a gram positive bacterial infection.

In one embodiment, the bacterial infection is an infection of Staphylococcus aureus, Hemolytic Stept, P, acnes, Clostridium, Bacillus, and Listeria. In one embodiment, the Staphylococcus aureus is Methicillin-resistant Staphylococcus aureus (MRSA).

In one embodiment, the methods of the invention treat Gram positive skin and soft tissue infections. For example, in one embodiment, Gram positive skin and soft tissue infections include, but are not limited to, Cellulitis, Impetigo, Erysipelas, Boils, Acne, Abscesses, Epidermolysis bullosa, Bullous diseases including those related to autoimmune disease such as pemphigoid, Toxic epidermal necrolysis, Infections secondary to environmental allergens (e.g., poison ivy) or drug reactions, Staphylococcal scalded skin syndrome.

In one embodiment, the method comprises administering to the subject an effective amount of Cannabidiol (CBD) or a CBD derivative, and Cannabinol (CBN) or a CBN derivative.

In one embodiment, the method comprises administering to the subject an effective amount of CBD or a cm) derivative and Cannabigerol (CBG) or a CBG derivative.

In one embodiment, the method comprises administering to the subject an effective amount of CBN or a CBN derivative and CBG or a CBG derivative.

In one embodiment, the method comprises administering to the subject an effective amount of CBD or a CBD derivative, CBN or a CBN derivative, and CBG or a CBG derivative.

Exemplary CBG derivatives include, but are not limited to Tetrahydrocannabigerol, 6,7-Epoxycannabigerol, Cannabigerolic acid, Tetrahydrocannabigerolic acid, (Z)-Cannabigerorcin, and Methyl tetrahydrocannabigerol.

Exemplary CBN derivatives include, but are not limited to, 8-Hydroxycannabinol, Cannabinolic acid, and Cannabinol acetate.

Exemplary CBD derivatives include, but are not limited to, Cannabidiolic acid, Monomethoxy cannabidiol, Cannabielsoin, Cannabichromene, and Cannabichromeorcin.

In one embodiment, the composition further comprises Δ8-Tetrahydrocannabinol (AS-THC) or a Δ8-THC derivative, Exemplary Δ8-THC derivatives include, but are not limited to, 2-dimethylamino-Δ8 THC, 2,8-dinitro-Δ8 THC, 2-butylamino-Δ8 THC, and Δ8-Tetrahydrocannabinol quinone.

One of skill in the art will appreciate that the cannabinoids of the invention can be administered singly or in any combination. Further, the cannabinoids of the invention can be administered singly or in any combination in a temporal sense, in that they may be administered concurrently, or before, and/or after each other. One of ordinary skill in the art will appreciate, based on the disclosure provided herein, that the cannabinoids compositions of the invention can be used to prevent or to treat a bacterial infection, and that a cannabinoid composition can be used alone or in any combination with another modulator to affect a therapeutic result. In various embodiments, any of the cannabinoid compositions of the invention described herein can be administered alone or in combination with other modulators of other molecules associated with bacterial infections or diseases/disorders associated with bacterial infections.

The method comprises administering a combination of cannabinoids in any suitable ratio. For example, in one embodiment, the method comprises administering two individual cannabinoids at a 1:1 ratio. In one embodiment, the method comprises administering three individual cannabinoids at a 1:1:1 ratio. However, the method is not limited to any particular ratio. Rather any ratio that is shown to be effective is encompassed.

In one embodiment, the method further comprises administering an additional therapeutic agent, In one embodiment, the additional therapeutic agent is an antibacterial agent.

In one embodiment, the method comprises administering to the subject an effective amount of Cannabidiol (CBD) or a CBD derivative, Cannabinol (CBN) or a CBN derivative, and an additional therapeutic agent.

In one embodiment, the method comprises administering to the subject an effective amount of CBD or a cm) derivative, Cannabigerol (CBG) or a CBG derivative, and an additional therapeutic agent.

In one embodiment, the method comprises administering to the subject an effective amount of CBN or a CBN derivative, CBG or a CBG derivative, and an additional therapeutic agent.

In one embodiment, the method comprises administering to the subject an effective amount of CBD or a CBD derivative, CBN or a CBN derivative. CBG or a CBG derivative, and an additional therapeutic agent.

In one embodiment, the additional therapeutic is selected from the group consisting of daptomycin (Cubicin), levolloxacin, doxycycline, neomycin, clindamycin, minocycline, gentamycin, rifampin, chlorhexidine, chloroxylenol, methylisothizolone, thymol, α-terpineol, cetylpyridinium chloride, hexachlorophene, triclosan, nitrofurantoin, erythromycin, nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, linexolid, temafloxacin, tosufloxacin, clinafloxacin, sulbactam, clavulanic acid, amphotericin B, fluconazole, itraconazole, ketoconazole, nystatin, penicillins, cephalosporins, carbepenems, beta-lactams antibiotics, aminoglycosides, macrolides, lincosamides, glycopeptides, tetracylines, chloramphenicol, quinolones, fucidines, sulfonamides, trimethoprims, rifamycins, oxalines, streptogramins, lipopeptides, ketolides, polyenes, azoles, echinocandines, and any combination thereof.

One of skill in the art will appreciate that the one or more cannabinoids and one or more additional therapeutics can be administered singly or in any combination. Further, the one or more cannabinoids and one or more additional therapeutics can be administered singly or in any combination in a temporal sense, in that they may be administered concurrently, or before, and/or after each other. One of ordinary skill in the art will appreciate, based on the disclosure provided herein, that the one or more cannabinoids and one or more additional therapeutics can be used to prevent or to treat a bacterial infection, and that the one or more cannabinoids and one or more additional therapeutics can be used alone or in any combination with another modulator to affect a therapeutic result.

The method comprises administering a combination of one or more cannabinoids and one or more additional therapeutics in any suitable ratio. For example, in one embodiment, the method comprises administering two individual cannabinoids and an additional therapeutic at a 1:1:1 ratio, In one embodiment, the method comprises administering three individual cannabinoids and an additional therapeutic at a 1:1:1:1 ratio. However, the method is not limited to any particular ratio. Rather any ratio that is shown to be effective is encompassed.

In one embodiment, the invention provides a method of preventing or reducing the growth or proliferation of microorganisms and/or biofilm-embedded microorganisms on at least one surface. In one embodiment, the method comprises the steps of: providing at least one surface; providing a composition comprising at least two cannabinoids, and applying the composition to the at least one surface in an amount sufficient to prevent or reduce the growth or proliferation of microorganisms or biofilm-embedded microorganisms on the at least one surface.

In one embodiment, the surface is a subject's body. In another embodiment, the surface is at least one surface of a medical device. In another embodiment, the surface is a solid surface. In another embodiment, the surface is selected from the group consisting of a glass surface, a metal surface, a paper surface, or a polymer surface. In one embodiment, the surface is the surface of a household item or a medical instrument. In one embodiment, the composition sterilizes the surface.

In one embodiment, the composition comprises an effective amount of Cannabidiol (CBD) or a CBD derivative, and Cannabinol (CBN) or a CBN derivative. In one embodiment, the composition comprises an effective amount of CBD or a CBD derivative and Cannabigerol (CBG) or a CBG derivative. In one embodiment, the composition comprises an effective amount of CBN or a CBN derivative and CBG or a CBG derivative. In one embodiment, the composition comprises an effective amount of CBD or a CBD derivative, CBN or a CBN derivative, and CBG or a CBG derivative.

In one embodiment, the composition further comprises an additional therapeutic. For example, in one embodiment, the composition further comprises an additional antibacterial agent. In one embodiment, the method further comprises providing a second composition comprising an additional therapeutic. In one embodiment, the method further comprises providing a second composition comprising an additional antibacterial agent.

In one embodiment, the invention includes a method comprising administering a combination of compounds described herein. in certain embodiments, the method has an additive effect, wherein the overall effect of the administering a combination of compounds is approximately equal to the sum of the effects of administering each individual compound. In other embodiments, the method has a synergistic effect, wherein the overall effect of administering a combination of compounds is greater than the sum of the effects of administering each individual compound.

### Pharmaceutical Compositions and Formulations

The invention also encompasses the use of pharmaceutical compositions of the invention or salts thereof to practice the methods of the invention. Such a pharmaceutical composition may consist of at least one cannabinoid composition of the invention or a salt thereof in a form suitable for administration to a subject, or the pharmaceutical composition may comprise at two cannabinoids of the invention or a salt thereof, and one or more pharmaceutically acceptable carriers, one or more additional ingredients, or some combination of these. The cannabinoids may be present in the pharmaceutical composition in the form of a physiologically acceptable salt, such as in combination with a physiologically acceptable cation or anion, as is well known in the art.

In an embodiment, the pharmaceutical compositions useful for practicing the methods of the invention may be administered to deliver a dose of between 1 rig/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, or any other tissue of a mammal Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art, and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful iter chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these, Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

In one embodiment, the formulation is a scaffold or substrate composition comprising a cannabinoid composition of the invention. For example, in one embodiment, a cannabinoid composition of the invention is within a scaffold. In another embodiment a cannabinoid composition of the invention is applied to the surface of a scaffold. The scaffold of the invention may be of any type known in the art. Non-limiting examples of such a scaffold includes a, hydrogel, electrospun scaffold, foam, mesh, sheet, patch, and sponge.

(a) Hydrogels

In one embodiment, the present invention provides a hydrogel comprising a cannabinoid composition of the invention. Hydrogels can generally absorb a great deal of fluid and, at equilibrium, typically are composed of 60-90% fluid and only 10-30% polymer. In a preferred embodiment, the water content of hydrogel is about 70-80%. Hydrogels are particularly useful due to the inherent biocompatibility of the cross-linked polymeric network (Hill-West, et al.,1994, Proc. Natl. Sci. USA 91:5967-5971). Hydrogel biocompatibility may be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27).

In one embodiment, the composition comprises a hydrogel comprising a cannabinoid composition of the invention. A cannabinoid hydrogel may comprise one or more other biopolymer or synthetic polymer. The hydrogels may be prepared by crosslinking hydrophilic biopolymers or synthetic polymers. Examples of the hydrogels formed from physical or chemical crosslinking of hydrophilic biopolymers, include but are not limited to, hyaluronans, chitosans, alginates, collagen, dextran, pectin, carrageenan, polylysine, gelatin or agarose, (see.: W. E. Hernnink and C. F. van Nostrum, 2002, Adv. Drug Del. Rev. 54, 13-36 and A. S. Hoffman, 2002, Adv. Drug Del. Rev. 43, 3-12). These materials consist of high-molecular weight backbone chains made of linear or branched polysaccharides or polypeptides. Examples of hydrogels based on chemical or physical crosslinking synthetic polymers include but are not limited to (meth)acrylate-oligolactide-PEO-oligolactide-(meth)acrylate, polyethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), polyethylene glycol) diacrylate (PEGDA), etc. (see A. S Hoffman, 2002Adv. Drug Del. Rev, 43, 3-12).

In certain embodiments, the hydrogel is modified to comprise one or more therapeutic agents, such as cannabinoids. Hydrogels may be modified with functional groups for covalently attaching a variety of compounds such as cannabinoids. In one embodiment, compounds, such as therapeutic agents, may be incorporated into the hydrogel matrix. Exemplary compounds include, but are not limited to, CBD or a CBD derivative, CBN or a CBN derivative, and CBG Or a CBG derivative.

Additional therapeutic agents which may be incorporated into the hydrogel scaffold include, but are not limited to, analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents. It is contemplated that linkage of the therapeutic agent to the matrix may be via a protease sensitive linker or other biodegradable linkage.

In certain embodiments, one or more multifunctional cross-linking agents known in the art may be utilized as reactive moieties that covalently link biopolymers or synthetic polymers.

Electrospun Scaffolds

In one embodiment, one or more cannabinoids or may be incorporated into nanofibrous biocompatible electrospun matrices. In some embodiments, cannabinoids may be blended with a synthetic polymer, such as polyethylene oxide) (PEO) to produce a tissue engineering scaffold.

The scaffolds of the invention may be produced in a variety of ways. In an exemplary embodiment, the scaffold may be produced by electrospinning. Electrospinning is an atomization process of a conducting fluid which exploits the interactions between an electrostatic field and the conducting fluid. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. As it reaches a grounded target, the material may be collected as an interconnected web containing relatively fine, i.e. small diameter, fibers. The resulting films (or membranes) from these small diameter fibers have very large surface area to volume ratios and small pore sizes. A detailed description of electrospinning apparatus is provided in Zong, et al., 2002 Polymer 43: 4403-4412; Rosen et al., 1990 Ann Plast Surg 25: 375-87; Kim, K., Biomaterials 2003, 24: 4977-85; Zong, X., 2005 Biomaterials 26: 5330-8. After electrospinning, extrusion and molding may be utilized to further fashion the polymers. To modulate fiber organization into aligned fibrous polymer scaffolds, the use of patterned electrodes, wire drum collectors, or post-processing methods such as uniaxial stretching has been successful. Zong, X., 2005 Biomaterials 26: 5330-8; Datta, P., 2004 Nano Lett 4: 2215-2218; Li, D., 2005 Nano Lett 5: 913-6. Methods to produce a solution comprising one or more cannabinoids suitable for electrospinning are well-known in the art.

The invention also includes combinations of natural materials, combinations of synthetic materials, and combinations of both natural and synthetic materials, For example, the cannabinoids may be combined with natural materials, synthetic materials, or both natural and synthetic materials to produce the scaffolds of the invention. Examples of combinations include, but are not. limited to: blends of different types of collagen (e.g. Type I with Type II, Type I with Type III, Type II with Type III, etc.); blends of one or more types of collagen with fibrinogen, thrombin, elastin, PGA, PLA, and polydioxanone; and blends of fibrinogen with one or more types of collagen, thrombin, elastin, PGA, PLA, and polydioxanone.

In embodiments in which the matrix contains substances that are to be released from the matrix, incorporating electroprocessed synthetic components, such as biocompatible substances, can modulate the release of substances from an electroprocessed composition. For example, layered or laminate structures may be used to control the substance release profile, Unlayered structures may also be used, in which case the release is controlled by the relative stability of each component of the construct. For example, layered structures composed of alternating electroprocessed materials are prepared by sequentially electroprocessing different materials onto a target. The outer layers are, for example, tailored to dissolve faster or slower than the inner layers. Multiple agents may be delivered by this method, optionally at different release rates. Layers may be tailored to provide a complex, multi-kinetic release profile of a single agent over time, Using combinations of the foregoing provides for release of multiple substances released, each with its own profile. Complex profiles are possible.

In some embodiments, the electroprocessed material itself may provide a therapeutic effect. For example, electroprocessed cannabinoids provides a therapeutic effect in treating or preventing bacterial infections.

(c) Method for Forming Matrices or Scaffolds

A biocompatible scaffold may be shaped using methods such as, for example, solvent casting, compression molding, filament drawing, meshing, leaching, weaving, foaming, electrospinning and coating, In solvent. casting, a solution of one or more proteins in an appropriate solvent, is cast as a branching pattern relief structure. After solvent evaporation, a thin film is obtained. In compression molding, a polymer is pressed at pressures up to 30,000 pounds per square inch into an appropriate pattern. Filament drawing involves drawing from the molten polymer and meshing involves forming a mesh by compressing fibers into a felt-like material. In leaching, a solution containing two materials is spread into a shape close to the final form of the artificial organ. Next a solvent is used to dissolve away one of the components, resulting in pore formation. (See U.S. Pat. No, 5,514,378 to Mikos).

The scaffold may be shaped into any number of desirable configurations to satisfy any number of overall system, geometry or space restrictions. For example, in the use of the scaffold for bladder, urethra, valve, or blood vessel reconstruction, the matrix or scaffold may be shaped to conform to the dimensions and shapes of the whole or a part of the tissue. The scaffold may be shaped in different sizes and shapes to conform to the organs of differently sized patients. The matrix or scaffold may also be shaped in other fashions to accommodate the special needs of the patient.

### Administration/Dosing

The regimen of administration may affect What constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation,

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range tor a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Cannabinoids of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 ing to about 2,500 mg, about 20 mg to about 2,000 mg, about 25 mg to about 1,500 mg, about 50 mg to about 1,000 tug, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a cannabinoids of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second cannabinoids as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Controlled- or sustained-release formulations of a pharmaceutical composition of the invention may be made using conventional technology, using for example proteins equipped with sensitive domains or protease-cleavable fragments. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel-caps, and caplets, which are adapted for controlled-release are encompassed by the present invention.

Advantages of controlled-release formulations include extended activity of the drug, reduced dosage frequency, and increased subject compliance. In addition, controlled-release formulations can be used to affect the time of onset of action or other characteristics, such as blood level of the drug, and thus can affect the occurrence of side effects. Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic: acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. Microencapsulation of recombinant proteins for sustained release has been successfully performed with human growth hormone (rhGH), interferon-(rhIFN-), interleukin-2, and MN rpg 120. Johnson et al., Nat. Med. 2: 795-799 (1996); Yasuda et al., Biomed Ther 27: 1221-1223 (1993); Hora et al., Bio/Technology 8: 755.-758 (1990); Cleland, “Design and Production of Single Immunization Vaccines Using Polylactide Polyglycolide Microsphere Systems,” in Vaccine Design: The Subunit and Adjuvant Approach, Powell and Newman, eds., (Plenum Press: New York, 1995), pp. 439-462; WO 97/03692; WO 96/40072; WO 96/07399; and U.S. Pat. No. 5,654,010.

The sustained-release formulations of these proteins may be developed using poly lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer”, in Biodegradable Polymers as Drug Delivery Systems (Marcel Dekker; New York, 1990), M. Chasin and R. Langer (Eds,) pp. 1-41.

While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

For sustained release, the compounds may be formulated with a suitable polymer or hydrophobic material that provides sustained release properties to the compounds. As such, the compounds for use the method of the invention may be administered in the form of microparticles, for example, by injection. or in the form of wafers or discs by implantation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

### Routes of Administration

Routes of administration of any of the compositions of the invention include oral, nasal parenteral, sublingual, transdermal, transmucosal (e.g., sublingual, lingual, (trans)buccal, (trans)urethral and (intra)nasal,), vaginal (e.g., trans- and perivaginally), subcutaneous, intravesical, intraduodenal, intragastrical, rectal, intra-peritoneal, subcutaneous, intramuscular, intradermal, ultra-arterial, intravenous, externaliternal pump, or topical.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, gels, lotions, oil-in-water or water-in-oil emulsions such as creams, ointments or pastes, and solutions or suspensions. Topically administrable formulations may, for example, comprise from about 1% to about 10% (w/v) active ingredient in a solvent, although the concentration of the active ingredient may be as high as the solubility limit of the active ingredient in the solvent. Formulations for topical administration may further comprise one or more of the additional ingredients described herein,

Enhancers of permeation may be used. These materials increase the rate of penetration of drugs across the skin Typical enhancers in the art include ethanol, glycerol monolaurate, PGML (polyethylene glycol monolaurate), dimethylsuifoxide (DMSO), and the like. Other enhancers include oleic acid, alcohol, ethoxydiglycol, laurocapram, alkanecarboxylic acids, dimethylsulfoxide, polar lipids, or N-methyl-2-pyrrolidone,

One acceptable vehicle for topical delivery of some of the compositions of the invention may contain liposomes. The composition of the liposomes and their use are known in the art (for example, see Constanza, U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum comeum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, Which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount. effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically-or naturally derived.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1

### Anti-Microbial Activity of Cannabinoids

The data presented herein demonstrates that cannabinoids and combinations of cannabinoids exert potent killing power against multiple species of MRSA. Table I demonstrates the IC₅₀, MIC and MBC of cannabinoids Tetrahydro CBG1, CBG, Tetrahydro CBG, CNB, CBD against MRS USA 300 compared to Mupirocin and Methicillin.

TABLE 1
MRS USA 300

| Compound | IC₅₀ (μM) | MIC (μM) | MBC (μM) |
|---|---|---|---|
| Tetrahydro CBG1 | 4.77 ± 0.64 | 9.46 ± 0 | 37.82 ± 0 |
| CBG | 2.43 ± 0.47 | 5.28 ± 2.27 | >63 |
| Tetrahydro CBG | 2.56 ± 0.31 | 6.49 ± 2.25 | >62 |
| CNB | 3.32 ± 0.35 | 8.05 ± 0 | 10.73 ± 4.64 |
| CBD | 2.99 ± 1.02 | 5.31 ± 2.29 | 10.59 ± 4.58 |
| Mupirocin | 0.08 ± 0.02 | 0.46 ± 0.22 | 3.76 ± 1.76 |
| Methicillin | 70.24 ± 15.48 | >131 | >131 |

Table 2 demonstrates the IC₅₀, MIC and MBC of cannabinoids Tetrahydro CBG1, CBG, Tetrahydro CBG, CNB, CBD against MRS 1708 (Mupirocin-resistant) compared to Mupirocin and Methicillin.

TABLE 2
MRS 1708 (Mupirocin-resistant)

| Compound | IC₅₀ (μM) | MIC (μM) | MBC (μM) |
|---|---|---|---|
| Tetrahydro CBG1 | 4.8 ± 0.79 | 9.46 ± 0 | 31.5 ± 10.93 |
| CBG | 2.34 ± 0.13 | 3.95 ± 0 | >63 |
| Tetrahydro CBG | 2.59 ± 0.62 | 5.21 ± 2.25 | 10.39 ± 4.49 |
| CNB | 2.77 ± 0.77 | 5.38 ± 7.32 | 6.7 ± 2.32 |
| CBD | 2.86 ± 0.83 | 5.31 ± 2.29 | 10.59 ± 4.58 |
| Mupirocin | >100 | >100 | >100 |
| Methicillin | 7.31 ± 0.97 | 65.72 ± 0 | 65.72 ± 0 |

Table 3 demonstrates the IC₅₀, MIC and MBC of cannabinoids Tetrahydro CBG1, CBG, Tetrahydro CBG, CNB, CBD against MRS 33591 compared to Mupirocin and Methicillin.

TABLE 3
MRS 33591

| Compound | IC₅₀ (μM) | MIC (μM) | MBC (μM) |
|---|---|---|---|
| Tetrahydro CBG1 | 5.26 ± 0.19 | 9.46 ± 0 | 31.5 ± 10.93 |
| CBG | 2.31 ± 0.09 | 3.95 ± 0 | 11.85 ± 5.59 |
| Tetrahydro CBG | 2.09 ± 0 | 3.9 ± 0 | 11.7 ± 5.52 |
| CNB | 2.48 ± 0.13 | 4.03 ± 0 | 13.43 ± 4.64 |
| CBD | 3.24 ± 1.05 | 6.61 ± 2.29 | 13.26 ± 4.58 |
| Mupirocin | 0.08 ± 0.02 | 0.44 ± 0.16 | 4.15 ± 2.6 |
| Methicillin | >131 | >131 | >131 |

Table 4 demonstrates the IC₅₀, MIC and MBC of cannabinoids Tetrahydro CBG1, CBG, Tetrahydro CBG, CNB, CBD against MRS USA 400compared to Mupirocin and Methicillin.

TABLE 4
MRS USA 400

| Compound | IC₅₀ (μM) | MIC (μM) | MBC (μM) |
|---|---|---|---|
| Tetrahydro CBG1 | 5.37 ± 0.42 | 9.46 ± 0 | 37.82 ± 0 |
| CBG | 2.72 ± 0.82 | 5.28 ± 2.27 | >63 |
| Tetrahydro CBG | 3.49 ± 0.5 | 7.8 ± 0 | 31.2 ± 0 |
| CNB | 4.16 ± 0.45 | 8.05 ± 0 | 18.78 ± 12.31 |
| CBD | 3.69 ± 0.51 | 7.95 ± 0 | 13.26 ± 4.58 |
| Mupirocin | 0.08 ± 0.04 | 0.32 ± 0 | 1.88 ± 0.88 |
| Methicillin | 8.78 ± 1.73 | 65.72 ± 0 | 109.54 ± 37.93 |

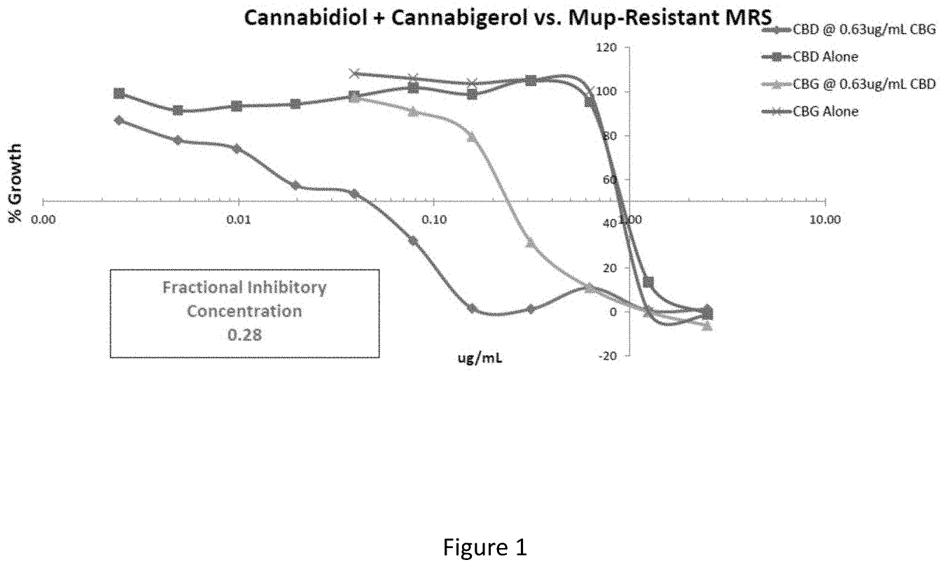

CBG, CBG, CBN and CBD were tested in combination with Mupirocin in Mupirocin-resistant MRS with no effect. CBG, CBN and CBD were tested in combination with Methicillin all 4 MRS strain with little to no effect (FIC>0.5). CBG, CBD and CBN were tested in combination with each other and synergy was seen in several instances (FIG. 1).

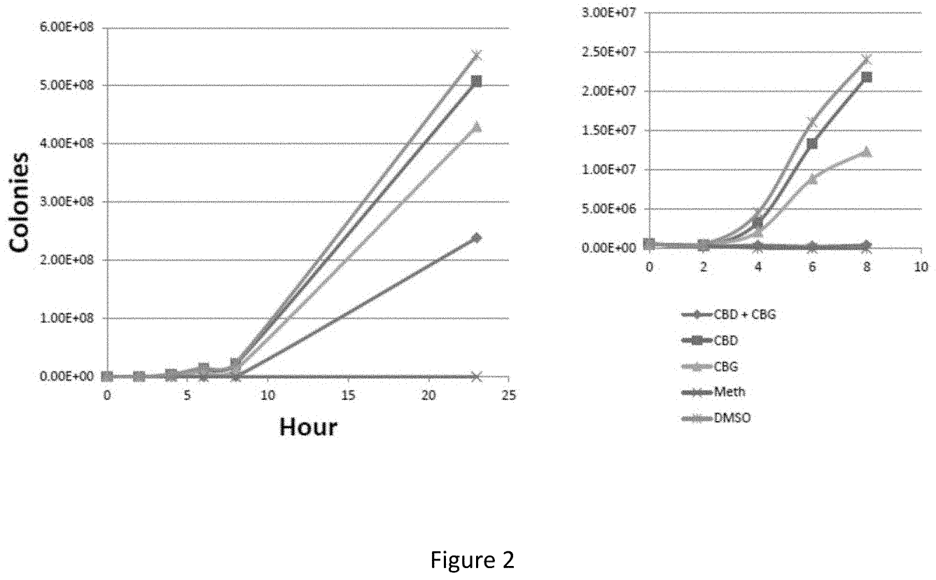

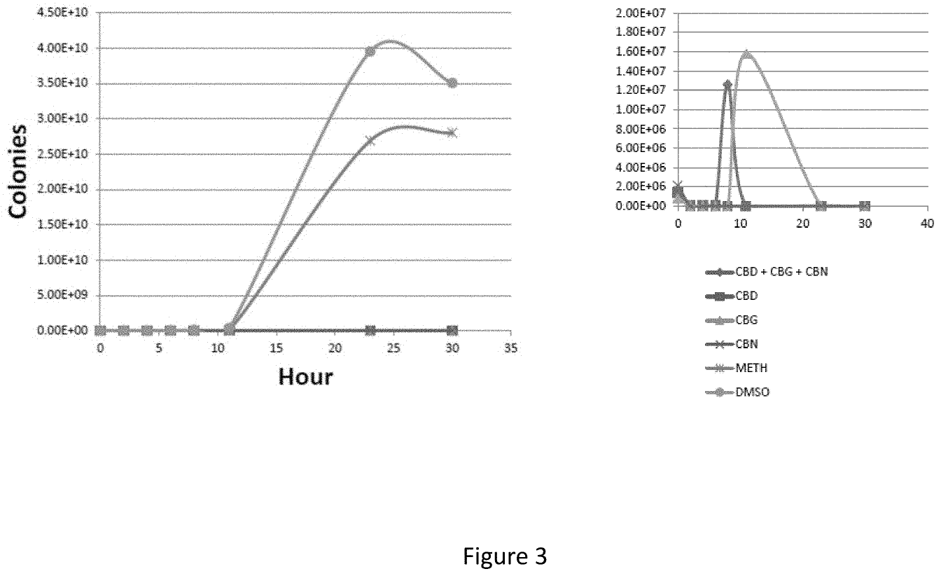

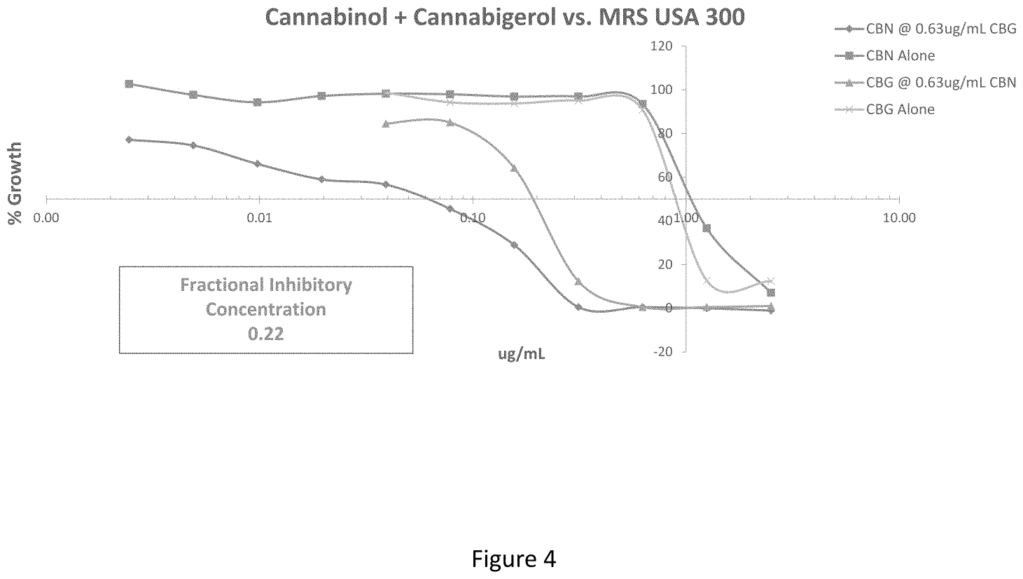

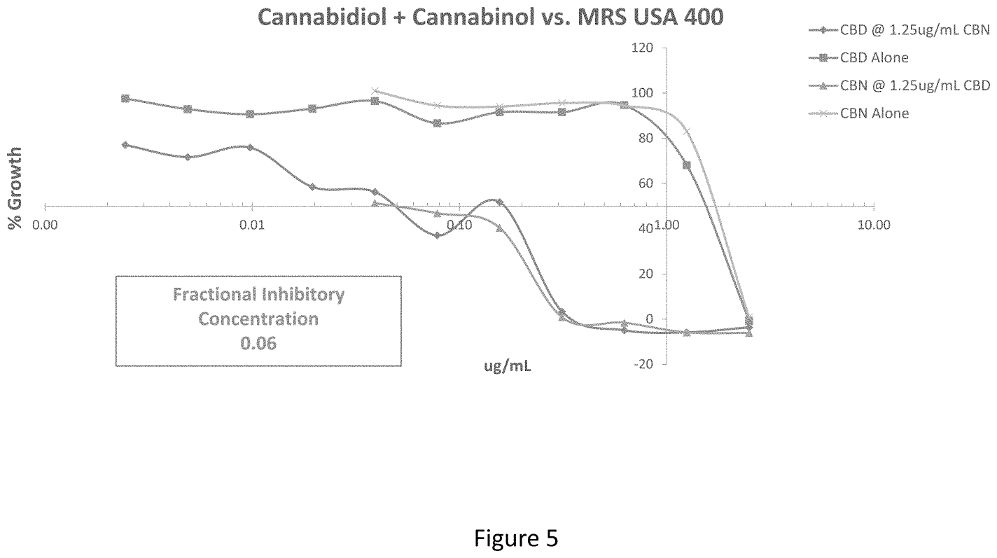

Based on checkerboard assay, selected CBD and CBG combinations were tested for time kill in Mupirocin-resistant MRS, Combinations showed improved effect compared to monotherapy (FIGS. 2). Triple therapy of CBG, CBD and CBN in MRS 35591 was tested at 5.3 μM each compared to each compound alone at 16 μM and methicillin at 131 μM (FIG. 3). The combination of CBN and CBG showed synergy against MRSA 300 strain (community-acquired MRSA) (FIG. 4). The combination of CBD and CBN showed synergy against MRS USA 400 (hospital-acquired MRSA) (FIG. 5).

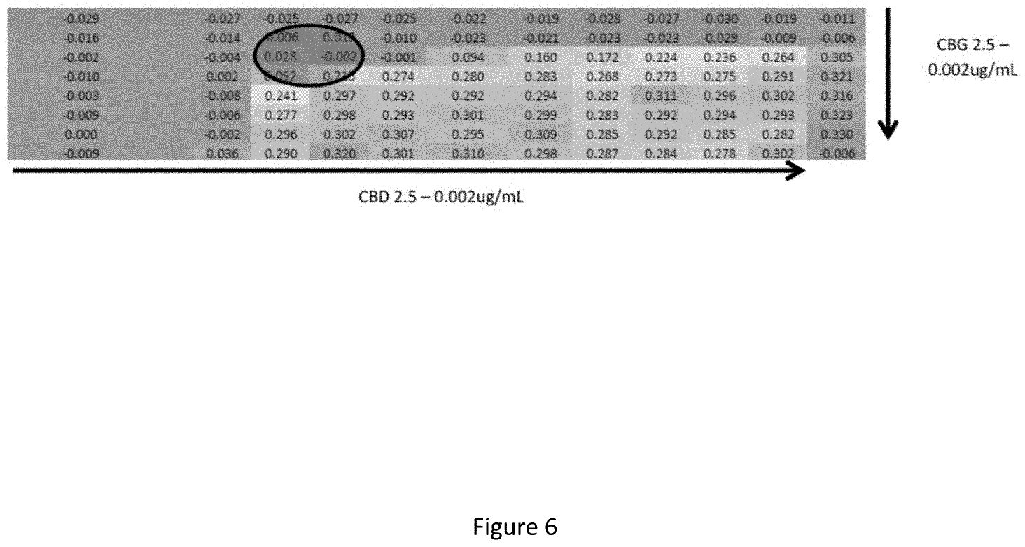

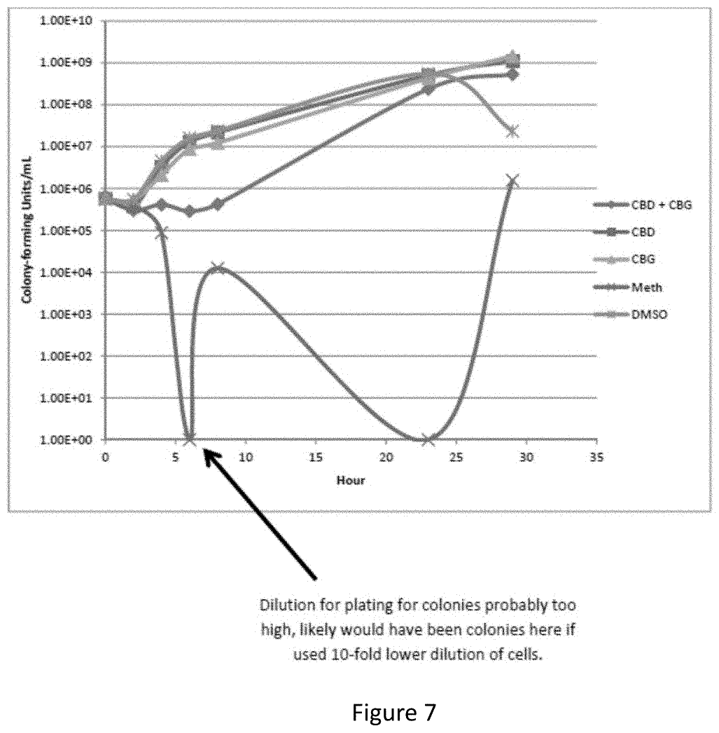

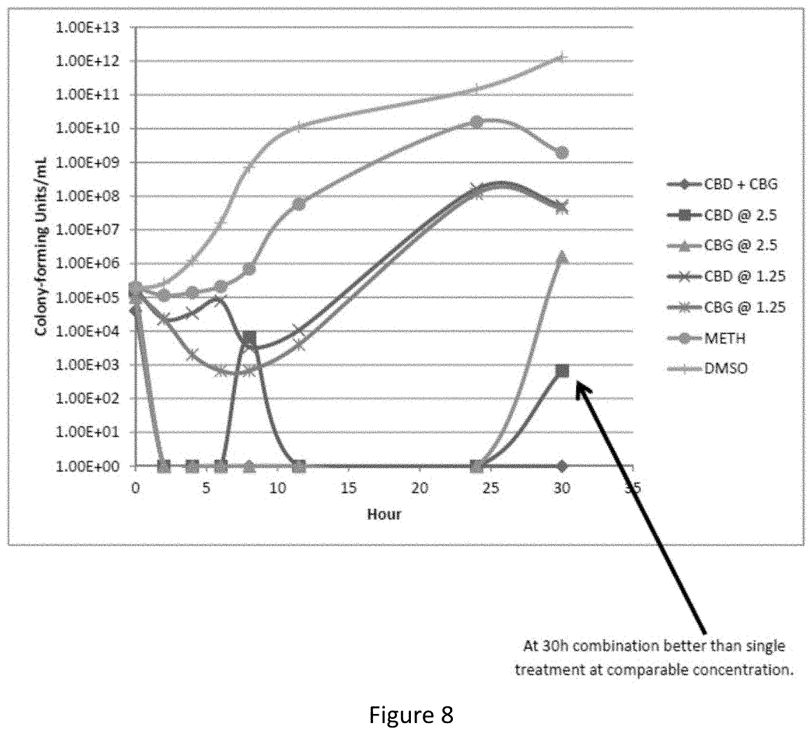

Checkerboard and Time Kill experiments were conducted on CBD and CBG to evaluate the potential bactericidal synergy in the Mupirocin-resistant strain of MRS (FIG. 6). The optimum combination from the checkefboard experiment was used in a time to kill experiment. The combination of CBD (0.5 μg/ml)+CBG (0.5 μg/ml) was compared to CBD (0.5 μg/ml) alone, CBG (0.5 μg/ml) alone, methicillin (50 μg/ml) and DMSO (FIG. 7). The combination of CBD (1.25 μg/ml)+CBG (1.25 μg/ml) was compared to CBD (2.5 μg/ml) alone, CBG (2.5 μg/ml) alone, CBD (1.25 μg/ml) alone, CBG (1.25 μg/ml) alone, methicillin (5 μg/ml) and DMSO (FIG. 8). At 30 hours, the combination was better than single treatment at comparable concentrations.

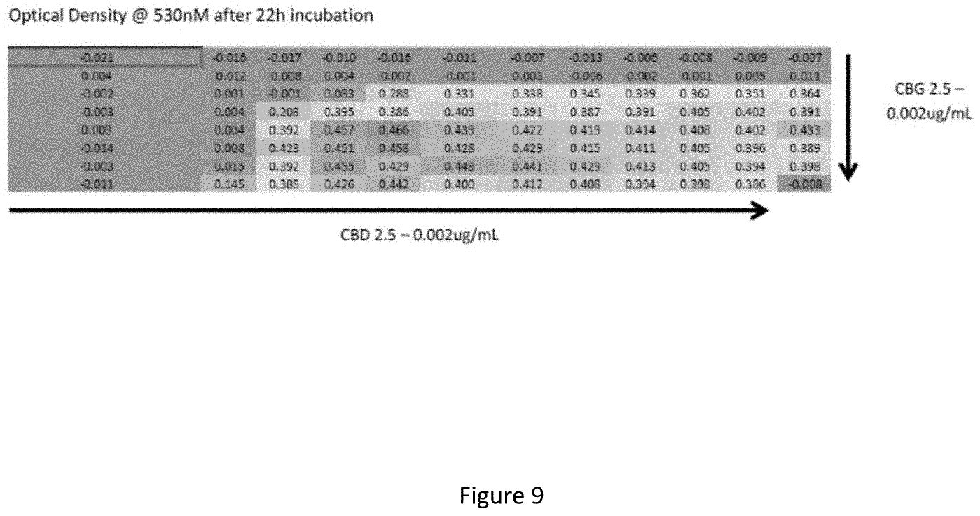

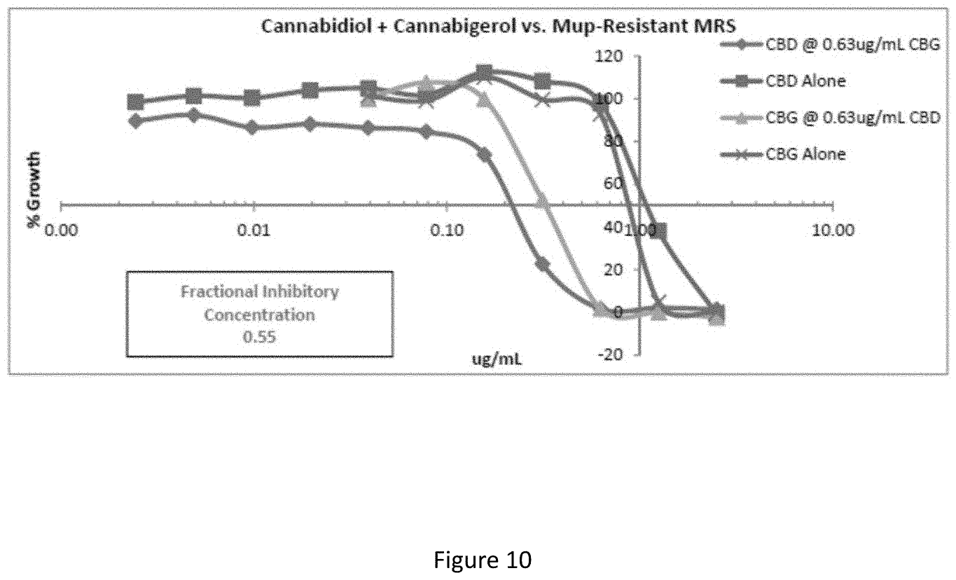

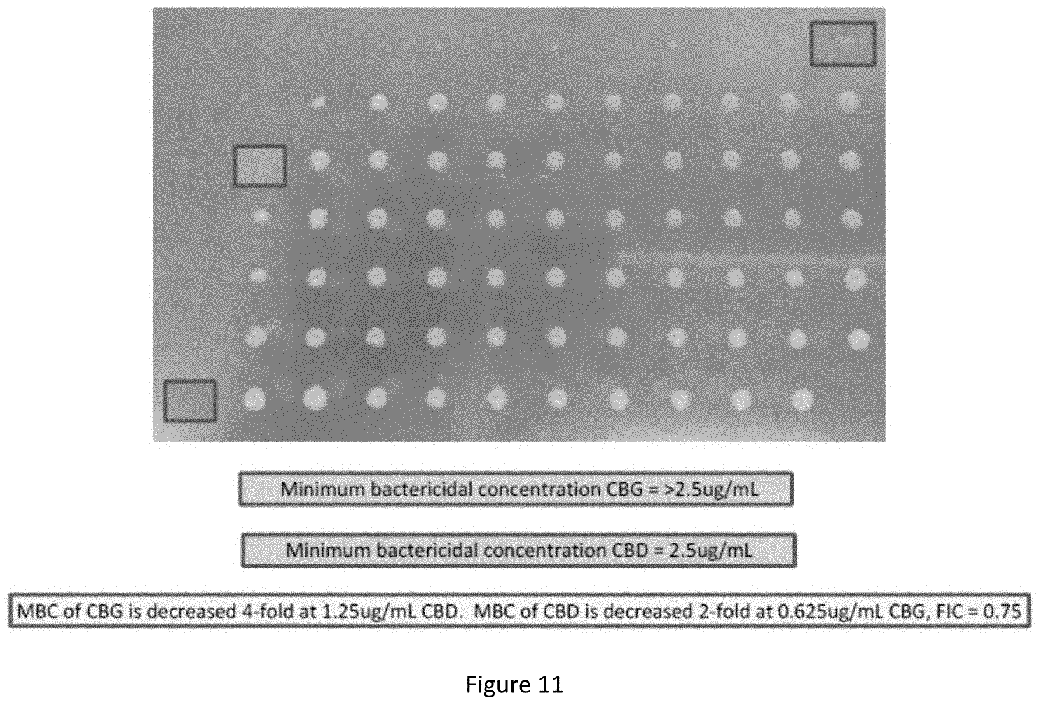

A second checkerboard experiment was carried out where after incubation, all e wells (10 uL) were pinned to agar to see bactericidal potential of all combinations. Pinning showed that there is less of a synergistic effect on bactericidal effect than minimum inhibitory concentrations (no growth via optical density) (FIGS. 9-11).

Table 5 shows the fractional inhibitory concentration of combinations of cannabinoids. Synergy<0.5.

TABLE 5

| Combination | FIC |
|---|---|
| Cannabigerol + Cannabichromene | 0.80 |
| Cannabidiol + Cannabichromene | 0.95 |
| Cannabinol + Cannabichromene | 1.03 |
| Cannabigerol + Cannabichromene-C1 | 1.19 |
| Cannabidiol + Cannabichromene-C1 | 1.37 |
| Cannabinol + Cannabichromene-C1 | 0.46 |

Table 6 demonstrates the IC₅₀, MIC and MBC against S. aureus 29213 of classes of cannabinoids compared to quinolones, beta lactams and monoxycarbolic acids.

TABLE 6
S. aureus 29213

| | | Conc. | | | |
|---|---|---|---|---|---|
| Sample Name | Class | (μg/mL) | IC₅₀ | MIC | MBC |
| Ciprofloxacin | Quinolone | 1-0.001 | 0.12 ± | 0.5 ± 0 | 0.67 ± |
| | | | 0.01 | | 0.29 |
| Methicillin | beta lactam | 50-0.049 | 0.65 ± | 2.6 ± 0.9 | 7 29 ± |
| | | | 0.11 | | 4.77 |
| Mupirocin | Monoxycarbolic | 5-0.005 | 0.04 ± | 0.39 ± | 1.25 ± 0 |
| | acid | | 0.01 | 0.33 | |
| 2-dimethylamino- Δ8 THC | Δ8 THC | 20-0.02 | NA | NA | NA |
| 2,8-dinitro- Δ8 THC | Δ8 THC | 20-0.02 | NA | NA | NA |
| 2-butylamino- Δ8 THC | Δ8 THC | 20-0.02 | NA | NA | NA |
| Cannabigerol | CBG | 20-0.02 | 0.75 ± | 1.25 ± 0 | NA |
| | | | 0.04 | | |
| Tetrahydrocannaboigerol | CBG | 20-0.02 | 0.7 ± | 1.25 ± 0 | NA |
| | | | 0.02 | | |
| 6,7-Epoxycannabigerol | CBG | 20-0.02 | 5.83 ± | 16.67 ± | NA |
| | | | 0.57 | 5.77 | |
| Cannabigerolic acid | CBG | 20-0.02 | 2.05 ± | 8.33 ± | NA |
| | | | 0.21 | 2.89 | |
| Tetrahydrocannabigerolic | CBG | 20-0.02 | 3.67 ± | 20 ± 0 | NA |
| acid | | | 0.52 | | |
| (Z)-Cannabigerorcin | CBG | 20-0.02 | 8.12 ± | 16.67 ± | NA |
| | | | 2.44 | 5.77 | |
| Methyl | CBG | 20-0.02 | 1.8 ± | 4.17 ± | 10 ± 0 |
| tetrahydrocannabigerol | | | 0.22 | 1.44 | |
| Cannabinol | CBN | 20-0.02 | 0.92 ± | 1.67 ± | NA |
| | | | 0.34 | 0.72 | |
| 8-Hydroxycannabinol | CBN | 20-0.02 | 1.2 ± | 5 ± 0 | NA |
| | | | 0.11 | | |
| Cannabinolic acid | CBN | 20-0.02 | NA | NA | NA |
| Cannabinol acetate | CBN | 20-0.02 | NA | NA | NA |
| Δ8-Tetrahydrocannabinol | Δ8 THC | 20-0.02 | 1.47 ± | 2.5 ± 0 | NA |
| | | | 0.02 | | |
| Δ8-Tetrahydrocannabinol | Δ8 THC | 20-0.02 | NA | NA | NA |
| quinone | | | | | |
| Cannabidiol | CBD | 20-0.02 | 1.16 ± | 2.08 ± | NA |
| | | | 0.25 | 0.72 | |
| Cannabidiolic acid | CBD | 20-0.02 | 6.83 ± | 16.67 ± | NA |
| | | | 3.4 | 5.77 | |
| Monomethoxy cannabidiol | CBD | 20-0.02 | NA | NA | NA |
| Cannabielsoin | CBD | 20-0.02 | 6.15 ± | 10 ± 0 | NA |
| | | | 0.14 | | |
| Cannabichromene | CBC | 20-0.02 | 6.96 ± | NA | NA |
| | | | 0.5 | | |
| Cannabichromeorcin | CBC | 20-0.02 | 6.94 ± | 10 ± 0 | NA |
| | | | 0.76 | | |
| Methyl | CBG | 20-0.02 | 1.53 ± | 25 ± 0 | 10 ± 0 |
| tetrahydrocannabigerol | | | 0.11 | | |

Table 7 demonstrates the IC₅₀, MIC and MBC against MRS 33591 of classes of cannabinoids compared to quinolones, beta lactams and monoxycarbolic acids,

TABLE 7
MRS 33591

| | | Conc. | | | |
|---|---|---|---|---|---|
| Sample Name | Class | (μg/mL) | IC₅₀ | MIC | MBC |
| Ciprofloxacin | Quinolone | 1-0.001 | 0.12 ± | 0.67 ± | 0.67 ± |
| | | | 0.01 | 0.29 | 0.29 |
| Methicillin | beta lactam | 50-0.049 | NA | NA | NA |
| Mupirocin | Monoxycarbotic | 5-0.005 | 0.04 ± | 0.22 ± | 2.08 ± |
| | acid | | 0.01 | 0.08 | 1.3 |
| 2-dimethylamino- Δ8 THC | Δ8 THC | 20-0.02 | NA | NA | NA |
| 2,8-dinitro- Δ8 THC | Δ8 THC | 20-0.02 | NA | NA | NA |
| 2-butylamino- Δ8 THC | Δ8 THC | 20-0.02 | NA | NA | NA |
| Cannabigerol | CBG | 20-0.02 | 0.73 ± | 1.25 ± 0 | 3.75 ± |
| | | | 0.03 | | 1.77 |
| Tetrahydrocannabigerol | CBG | 20-0.02 | 0.67 ± 0 | 1.25 ± 0 | 3.75 ± |
| | | | | | 1.77 |
| 6,7-Epoxycannabigerol | CBG | 20-0.02 | 6.73 ± | 13.33 ± | NA |
| | | | 0.53 | 5.77 | |
| Cannabigerolic acid | CBG | 20-0.02 | 2.29 ± | 13,33 ± | NA |
| | | | 0.46 | 5.77 | |
| Tetrahydrocannabigerolic | CBG | 20-0.02 | 4.33 ± | 20 ± 0 | NA |
| acid | | | 0.18 | | |
| (Z)-Cannabigerorcin | CBG | 20-0.02 | 9.48 ± | 20 ± 0 | NA |
| | | | 1.98 | | |
| Methyl | CBG | 20-0.02 | 1.83 ± | 4.17 ± | 15 ± 8.66 |
| tetrahydrocannabigerol | | | 0.55 | 1.44 | |
| Cannabinol | CBN | 20-0.02 | 0.77 ± | 1.25 ± 0 | 4.17 |
| | | | 0.04 | | 1.44 |
| 8-Hydroxycannabinol | CBN | 20-0.02 | 2.33 ± | 5 ± 0 | NA |
| | | | 0.21 | | |
| Cannabinolic acid | CBN | 20-0.02 | NA | NA | NA |
| Cannabinol acetate | CBN | 20-0.02 | NA | NA | NA |
| Δ8-Tetrahydrocannabinol | Δ8 THC | 20-0.02 | 1.43 ± | 2.5 ± 0 | 10 ± 0 |
| | | | 0.03 | | |
| Δ8-Tetrahydrocannabinol | Δ8 THC | 20-0.02 | NA | NA | NA |
| quinone | | | | | |
| Cannabidiol | CBD | 20-0.02 | 1.02 ± | 2.08 ± | 4.17 ± |
| | | | 0.33 | 0.72 | 1.44 |
| Cannabidiolic acid | CBD | 20-0.02 | 7.27 ± | 20 ± 0 | NA |
| | | | 1.69 | | |
| Monomethoxy cannabidiol | CBD | 20-0.02 | NA | NA | NA |
| Cannabielsoin | CBD | 20-0.02 | 6.26 ± | 10 ± 0 | NA |
| | | | 0.26 | | |
| Cannabichromene | CBC | 20-0.02 | 2.58 ± | 5 ± 0 | NA |
| | | | 0.13 | | |
| Cannabichromeorein | CBC | 20-0.02 | 5.56 ± | 10 ± 0 | 20 ± 0 |
| | | | 0.05 | | |
| Methyl | CBG | 20-0.02 | 1.39 ± | 2.5 ± 0 | 8.33 ± |
| tetrahydrocannabigerol | | | 0.05 | | 2.89 |

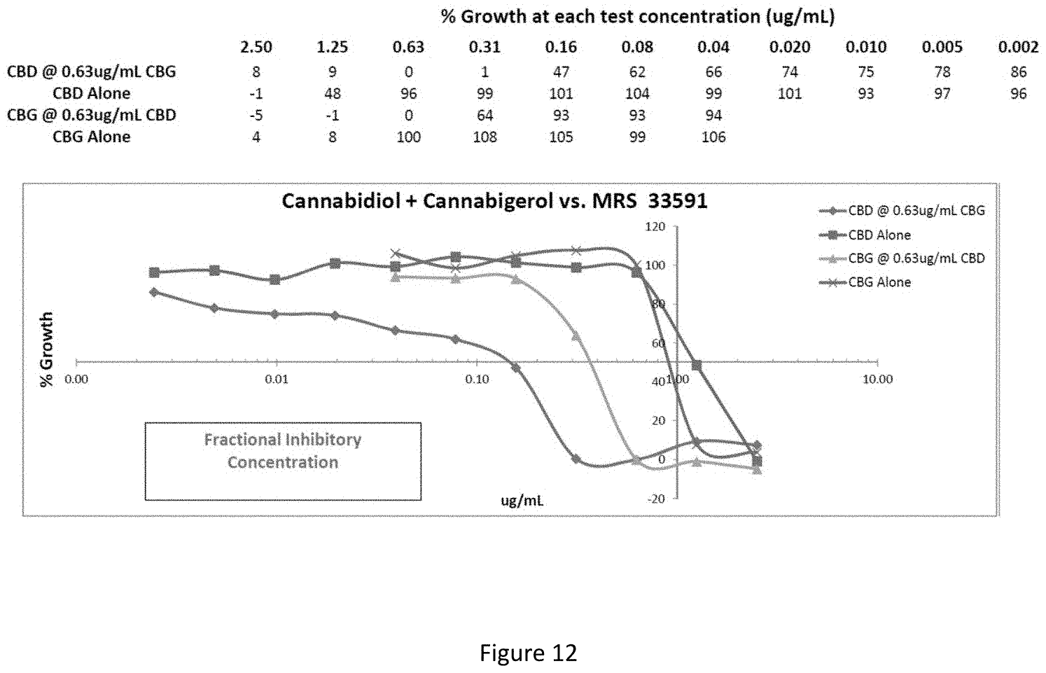

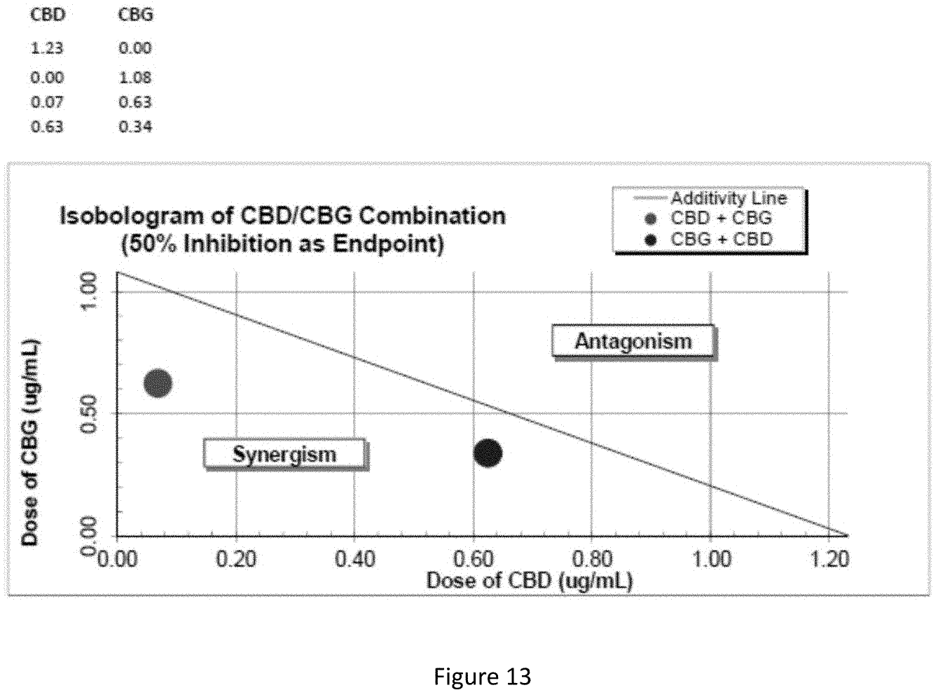

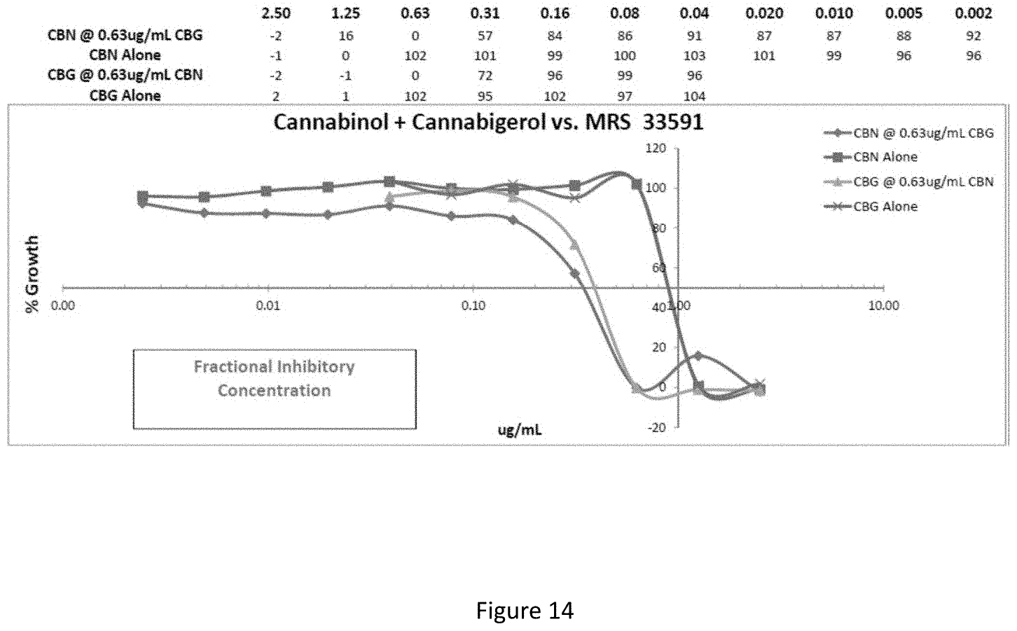

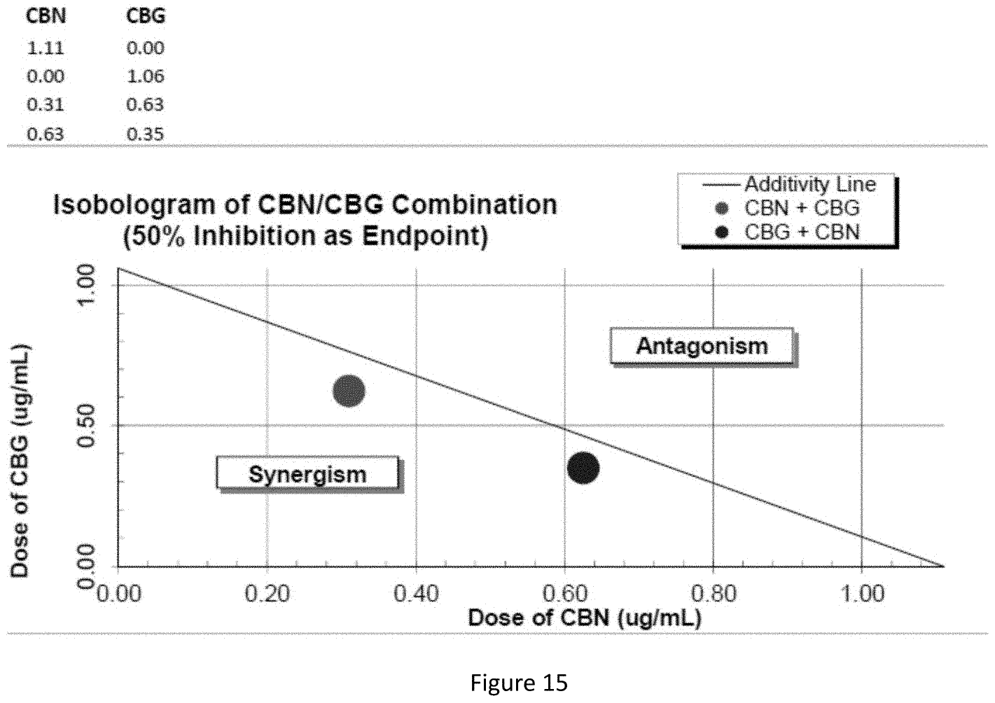

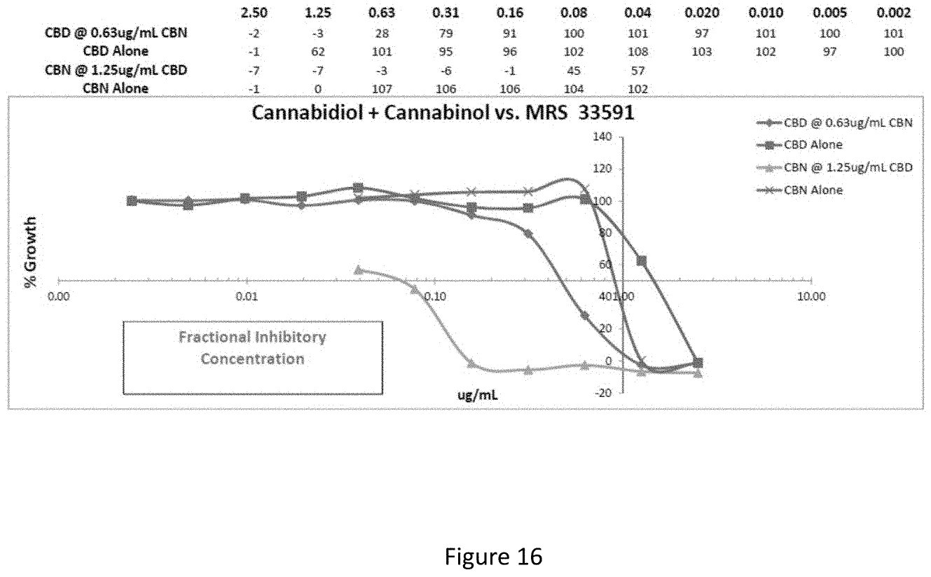

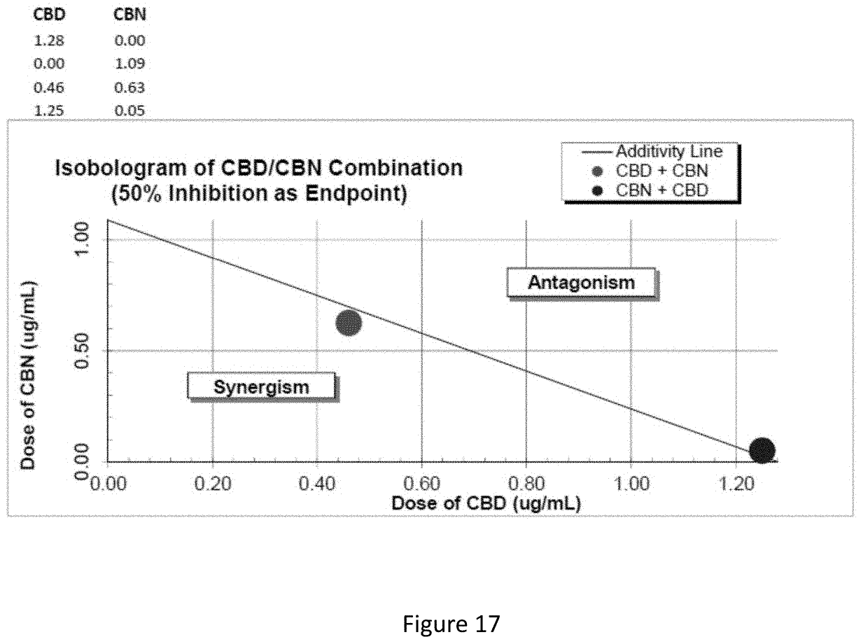

Table 8 demonstrates the IC₅₀s chosen for FICs against MRS33591. A summary of the analysis of combinations of cannabinoids against MRS 33591 is presented in FIGS. 12-17.

TABLE 8
MRS 33591: IC_{50S} chosen for FICs

| Cannabidiol (CBD) + | | Cannabinol (CBN) + | | Cannabidiol (CBD) + | |
|---|---|---|---|---|---|
| Cannabigerol (CBG) | IC50 | Cannabigerol (CBG) | IC50 | Cannabinol (CBN) | IC50 |
| CBD @ 2.5 ug/mL CBG | <0.002 | CBN @ 2.5 ug/mL CBG | <0.002 | CBD @ 2.5 ug/mL CBN | <0.002 |
| CBD @ 1.25 ug/mL CBG | <0.002 | CBN @ 1.25 ug/mL CBG | <0.002 | CBD @ 1.25 ug/mL CBN | <0.002 |
| CBD @ 0.63 ug/mL CBG | 0.069 | CBN @ 0.63 ug/mL CBG | 0.311 | CBD @ 0.63 ug/mL CBN | 0.462 |
| CBD @ 0.31 ug/mL CBG | 0.686 | CBN @ 0.31 ug/mL CBG | 0.752 | CBD @ 0.31 ug/mL CBN | 0.694 |
| CBD @ 0.16 ug/mL CBG | 0.901 | CBN @ 0.16 ug/mL CBG | 0.986 | CBD @ 0.16 ug/mL CBN | 0.733 |
| CBD @ 0.08 ug/mL CBG | 0.843 | CBN @ 0.08 ug/mL CBG | 1.008 | CBD @ 0.08 ug/mL CBN | 1.200 |
| CBD @ 0.04 ug/mL CBG | 0.975 | CBN @ 0.04 ug/mL CBG | 0.732 | CBD @ 0.04 ug/mL CBN | 1.266 |
| CBD @ 0 ug/mL CBG | 1.234 | CBN @ 0 ug/mL CBG | 1.107 | CBD @ 0 ug/mL CBN | 1.282 |
| CBG @ 2.5 ug/mL CBD | <0.039 | CBG @ 2.5 ug/mL CBN | <0.039 | CBN @ 2.5 ug/mL CBD | <0.039 |
| CBG @ 1.25 ug/mL CBD | <0.039 | CBG @ 1.25 ug/mL CBN | <0.039 | CBN @ 1.25 ug/mL CBD | 0.052 |
| CBG @ 0.63 ug/mL CBD | 0.341 | CBG @ 0.63 ug/mL CBN | 0.346 | CBN @ 0.63 ug/mL CBD | 0.501 |
| CBG @ 0.31 ug/mL CBD | 0.378 | CBG @ 0.31 ug/mL CBN | 0.654 | CBN @ 0.31 ug/mL CBD | 0.684 |
| CBG @ 0.16 ug/mL CBD | 0.618 | CBG @ 0.16 ug/mL CBN | 0.683 | CBN @ 0.16 ug/mL CBD | 0.707 |
| CBG @ 0.08 ug/mL CBD | 0.642 | CBG @ 0.08 ug/mL CBN | 0.693 | CBN @ 0.08 ug/mL CBD | 1.046 |
| CBG @ 0.04 ug/mL CBD | 0.650 | CBG @ 0.04 ug/mL CBN | 0.711 | CBN @ 0.04 ug/mL CBD | 1.098 |
| CBG @ 0.02 ug/mL CBD | 0.669 | CBG @ 0.02 ug/mL CBN | 0.700 | CBN @ 0.02 ug/mL CBD | 0.704 |
| CBG @ 0.01 ug/mL CBD | 0.663 | CBG @ 0.01 ug/mL CBN | 0.724 | CBN @ 0.01 ug/mL CBD | 0.913 |
| CBG @ 0.005 ug/mL CBD | 0.670 | CBG @ 0.005 ug/mL CBN | 0.697 | CBN @ 0.005 ug/mL CBD | 0.995 |
| CBG @ 0.002 ug/mL CBD | 0.773 | CBG @ 0.002 ug/mL CBN | 0.713 | CBN @ 0.002 ug/mL CBD | 0.858 |
| CBG @ 0 ug/mL CBD | 1.076 | CBG @ 0 ug/mL CBN | 1.055 | CBN @ 0 ug/mL CBD | 1.094 |

Table 9 demonstrates the IC₅₀, MIC and MBC against MRS USA 300 of classes of cannabinoids compared to quinolones, beta lactams and monoxycarbolic acids.

TABLE 9
MRS USA 300

| | | Conc. | | | |
|---|---|---|---|---|---|
| Sample Name | Class | (μg/mL) | IC₅₀ | MIC | MBC |
| Ciprofloxacin | Quinolone | 1-0.001 | 0.13 ± | 0.5 ± 0 | 0.67 ± |
| | | | 0.01 | | 0.29 |
| Methicillin | beta lactam | 50-0.049 | 26.72 ± | NA | NA |
| | | | 5.89 | | |
| Mupirocin | Monoxycarbolic | 5-0.005 | 0.04 ± | 0.23 ± | 1.85 ± |
| | acid | | 0.01 | 0.11 | 0.88 |
| 2-dimethylamino - Δ8 THC | Δ8 THC | 20-0.02 | NA | NA | NA |
| 2,8-dinitro- Δ8 THC | Δ8 THC | 20-0.02 | NA | NA | NA |
| 2-butylamino- Δ8 THC | Δ8 THC | 20-0.02 | NA | NA | NA |
| Cannabigerol | CBG | 20-0.02 | 0.77 ± | 1.67 ± | NA |
| | | | 0.15 | 0.72 | |
| Tetrahydrocannabigerol | CBG | 20-0.02 | 0.82 ± 0.1 | 2.08 ± | NA |
| | | | | 0.72 | |
| 6,7-Epoxycannabigerol | CBG | 20-0.02 | 7.53 ± | 20 ± 0 | NA |
| | | | 0.76 | | |
| Cannabigerolic acid | CBG | 20-0.02 | 1.52 ± | 8.33 ± | NA |
| | | | 0.18 | 2.89 | |
| Tetrahydrocannabigerolic | CBG | 20-0.02 | 2.98 ± | 20 ± 0 | NA |
| acid | | | 0.12 | | |
| (Z)-Cannabigerorcin | CBG | 20-0.02 | 6.46 ± | 16.67 ± | NA |
| | | | 1.34 | 5.77 | |
| Methyl | CBG | 20-0.02 | 1.76 ± | 4.17 ± | 13.33 ± |
| tetrahydrocannabigerol | | | 0.25 | 1.44 | 5.77 |
| Cannabinol | CBN | 20-0.02 | 1.03 ± | 2.5 ± 0 | 3.33 ± |
| | | | 0.11 | | 1.44 |
| 8-Hydroxycannabinol | CBN | 20-0.02 | 1.58 ± | 5 ± 0 | NA |
| | | | 0.12 | | |
| Cannabinolic acid | CBN | 20-0.02 | NA | NA | NA |
| Cannabinol acetate | CBN | 20-0.02 | NA | NA | NA |
| Δ8-Tetrahydrocannabinol | Δ8 THC | 20-0.02 | 1.91 ± | 5 ± 0 | NA |
| | | | 0.06 | | |
| Δ8-Tetrahydrocannabinol | Δ8 THC | 20-0.02 | NA | NA | NA |
| quinone | | | | | |
| Cannabidiol | CBD | 20-0.02 | 0.94 ± | 1.67 ± | 3.33 ± |
| | | | 0.32 | 0.72 | 1.44 |
| Cannabidiolic acid | CBD | 20-0.02 | 6.11 ± | 20 ± 0 | NA |
| | | | 2.26 | | |
| Monomethoxy cannabidiol | CBD | 20-0.02 | NA | NA | NA |
| Cannabielsoin | CBD | 20-0.02 | 5.49 ± | 10 ± 0 | 20 ± 0 |
| | | | 0.23 | | |
| Cannabichromene | CBC | 20-0.02 | 6.44 ± | NA | NA |
| | | | 0.62 | | |
| Cannabichromeorcin | CBC | 20-0.02 | 5.41 ± | 10 ± 0 | 13.33 ± |
| | | | 0.07 | | 5.77 |
| Methyl | CBG | 20-0.02 | 1.26 ± | 2.5 ± 0 | 10 ± 0 |
| tetrahydrocannabigerol | | | 0.17 | | |

Table 10 demonstrates the IC₅₀, MIC and MBC against MRS USA 400 of classes of cannabinoids compared to quinolones, beta lactams and monoxycafbolic acids.

TABLE 10
MRS USA 400

| | | Conc. | | | |
|---|---|---|---|---|---|
| Sample Name | Class | (μg/mL) | IC₅₀ | MIC | MBC |
| Ciprofloxacin | Quinolone | 1-0.001 | NA | NA | NA |
| Methicillin | beta lactam | 50-0.049 | 3.34 ± | | 41.67 ± |
| | | | 0.66 | 25 ± 0 | 14.43 |
| Mupirocin | Monoxycarbolic | 5-0.005 | 0.04 ± | 0.16 ± 0 | 0.94 ± 0.44 |
| | acid | | 0.02 | | |
| 2-dimethylamino- Δ8 THC | Δ8 THC | 20-0.02 | NA | NA | NA |
| 2,8-dinitro- Δ8 THC | Δ8 THC | 20-0.02 | NA | NA | NA |
| 2-butylamino- Δ8 THC | Δ8 THC | 20-0.02 | NA | NA | NA |
| Cannnabigerol | CBG | 20-0.02 | 0.86 ± | 1.67 ± | NA |
| | | | 0.26 | 0.72 | |
| Tetrahydrocannabigerol | CBG | 20-0.02 | 1.12 ± | 2.5 ± 0 | 10 ± 0 |
| | | | 0.16 | | |
| 6,7-Epoxycannabigerol | CBG | 20-0.02 | 8.67 ± | 20 ± 0 | NA |
| | | | 0.89 | | |
| Cannabigerolic acid | CBG | 20-0.02 | 1.67 ± | 5 ± 0 | NA |
| | | | 0.13 | | |
| Tetrahydrocannabigerolic | CBG | 20-0.02 | 2.79 ± | 13.33 ± | NA |
| acid | | | 0.26 | 5.77 | |
| (Z) Cannabigerorcin | CBG | 20-0.02 | 7.27 ± | 13.33 ± | NA |
| | | | 1.38 | 5.77 | |
| Methyl | CBG | 20-0.02 | 1.91 ± | 5.83 ± | 16.67 ± |
| tetrahydrocannabigerol | | | 0.48 | 3.82 | 5.77 |
| Cannabinol | CBN | 20-0.02 | 1.29 ± | 2.5 ± 0 | 5.83 ± 3.82 |
| | | | 0.14 | | |
| 8-Hydroxycannabinol | CBN | 20-0.02 | 1.82 ± | 5 ± 0 | NA |
| | | | 0.22 | | |
| Cannabinolic acid | CBN | 20-0.02 | NA | NA | NA |
| Cannabinol acetate | CBN | 20-0.02 | NA | NA | NA |
| Δ8-Tetrahydrocannabinol | Δ8 THC | 20-0.02 | 2.55 ± | 10 ± 0 | NA |
| | | | 0.05 | | |
| Δ8-Tetrahydrocannabinol | Δ8 THC | 20-0.02 | NA | NA | NA |
| quinone | | | | | |
| Cannabidiol | CBD | 20-0.02 | 1.16 ± | 2.5 ± 0 | 4.17 ± 1.44 |
| | | | 0.16 | | |
| Cannabidiolic acid | CBD | 20-0.02 | 4.99 ± | 13.33 ± | NA |
| | | | 1.02 | 5.77 | |
| Monomethoxy cannabidiol | CBD | 20-0.02 | NA | NA | NA |
| Cannabielsoin | CBD | 20-0.02 | 6.03 ± | 10 ± 0 | NA |
| | | | 0.26 | | |
| Cannabichromene | CBC | 20-0.02 | 7.28 ± | NA | NA |
| | | | 0.21 | | |
| Cannabichromeorcin | CBC | 20-0.02 | 5.9 ± | 10 ± 0 | 20 ± 0 |
| | | | 0.58 | | |
| Methyl | CBG | 20-0.02 | 1.42 ± | 2.5 ± 0 | 10 ± 0 |
| tetrahydrocannabigerol | | | 0.11 | | |

Table 11 demonstrates the 1030, MIC and MBC against Mupirocin-resistant MRS of classes of cannabinoids compared to quinolones, beta lactams and monoxycarbolic acids.

TABLE 11
Mupirocin-resistant MRS

| Sample Name | Class | IC₅₀ | MIC | AMC |
|---|---|---|---|---|
| Ciprofloxacin | Quinolone | NA | NA | NA |
| Methicillin | beta lactam | 2.78 ± 0.37 | 25 ± 0 | 25 ± 0 |
| Mupirocin | Monoxycarbolic acid | NA | NA | NA |
| 2-dimethylamino- Δ8 THC | Δ8 THC | NA | NA | NA |
| 2,8-dinitro- Δ8 THC | Δ8 THC | NA | NA | NA |
| 2-butylamino- Δ8 THC | Δ8 THC | NA | NA | NA |
| Cannabigerol | CBG | 0.74 ± 0.04 | 1.25 ± 0 | NA |
| Tetrahydrocannabigerol | CBG | 0.83 ± 0.2 | 1.67 ± 0.72 | 3.33 ± 1.44 |
| 6,7-Epoxycannabigerol | CBG | 7.74 ± 1.17 | 20 ± 0 | 20 ± 0 |
| Cannabigerolic acid | CBG | 1.55 ± 0.27 | 6.67 ± 2.89 | NA |
| Tetrahydrocannabigerolic acid | CBG | 3.24 ± 0.13 | 20 ± 0 | NA |
| (Z)-Cannabigerorcin | CBG | 6.93 ± 1.54 | 13.33 ± 5.77 | NA |
| Methyl tetrahydrocannabigerol | CBG | 1.65 ± 0.25 | 3.33 ± 1.44 | 13.33 ± 5.77 |
| Cannabinol | CBN | 0.86 ± 0.24 | 1.67 ± 0.72 | 2.08 ± 0.72 |
| 8-Hydroxycannabinol | CBN | 2.15 ± 0.22 | 6.67 ± 2.89 | NA |
| Cannabinolic acid | CBN | NA | NA | NA |
| Cannabinol acetate | CBN | NA | NA | NA |
| Δ8-Tetrahydrocannabinol | Δ8 THC | 1.82 ± 0.08 | 5 ± 0 | 10 ± 8.66 |
| Δ8-Tetrahydrocannabinol quinone | Δ8 THC | NA | NA | NA |
| Cannabidiol | CBD | 0.9 ± 0.26 | 1.67 ± 0.72 | 13.33 ± 1.44 |
| Cannabidiolic acid | CBD | 5.57 ± 0.68 | 13.33 ± 5.77 | NA |
| Monomethoxy cannabidiol | CBD | NA | NA | NA |
| Cannabielsoin | CBD | 6.03 ± 0.49 | 10 ± 0 | 20 ± 0 |
| Cannabichromene | CBC | 5.04 ± 0.55 | NA | NA |
| Cannabichromeorcin | CBC | 5.57 ± 0.27 | 10 ± 0 | 13.33 ± 5.77 |
| Methyl tetrahydrocannabigerol | CBG | 1.27 ± 0.21 | 2.5 ± 0 | 8.33 ± 2.89 |

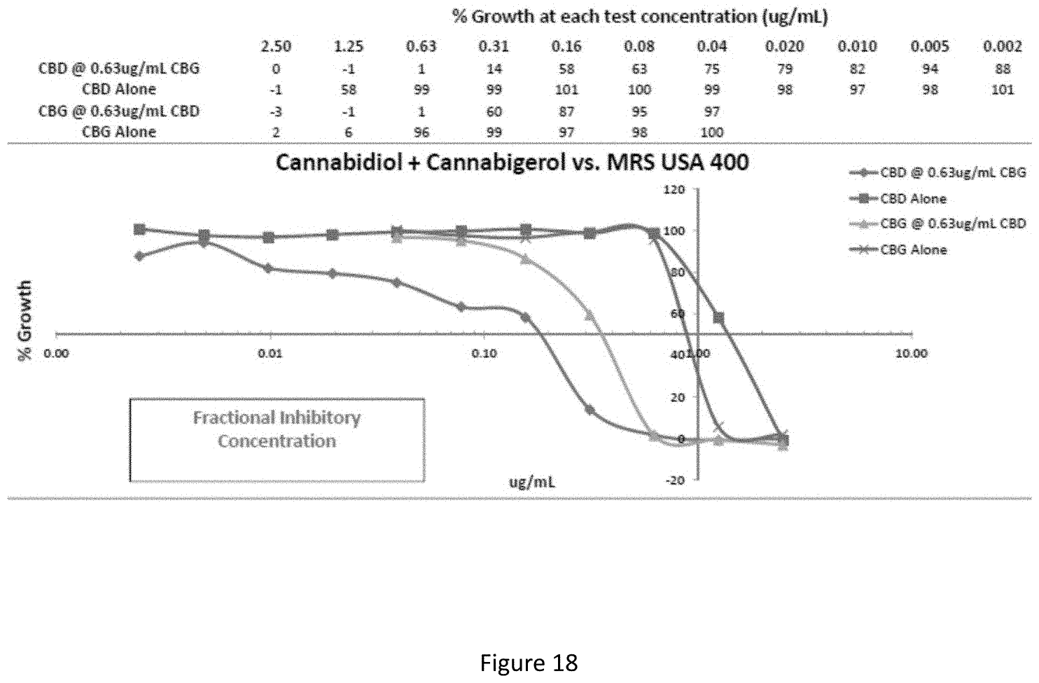

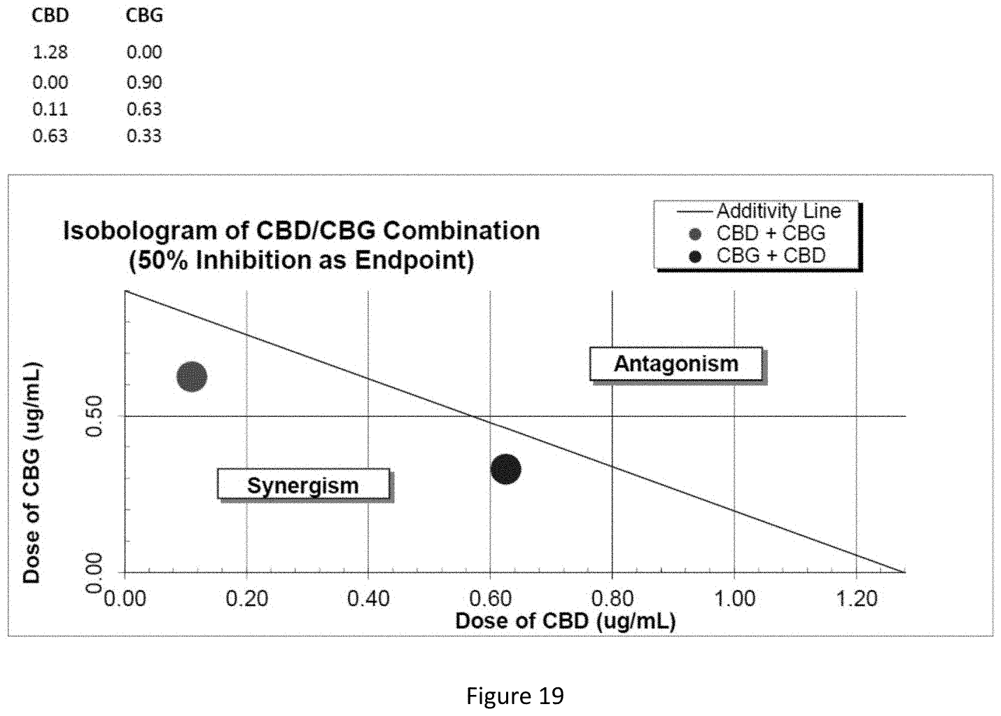

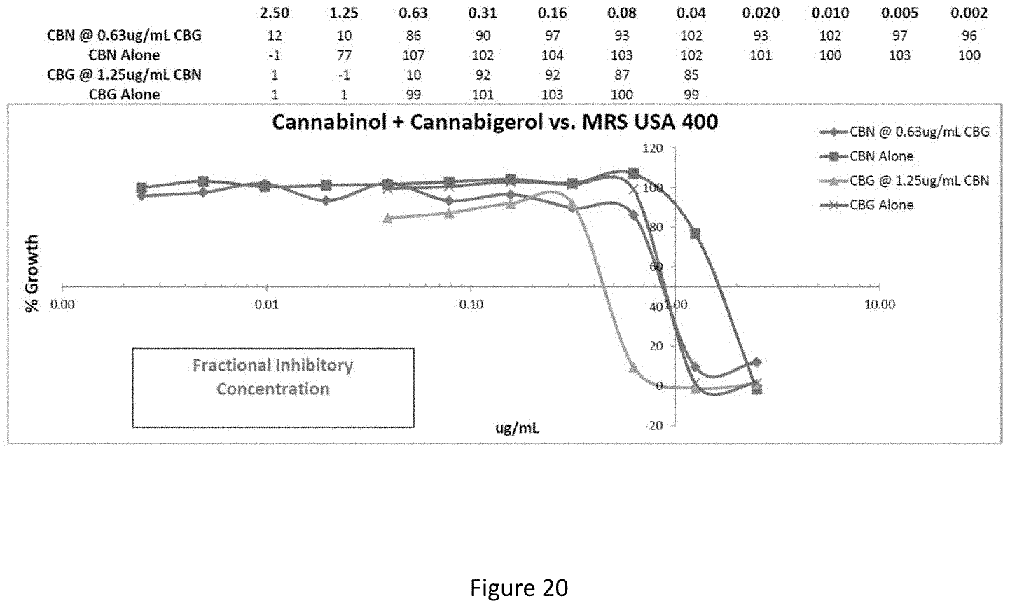

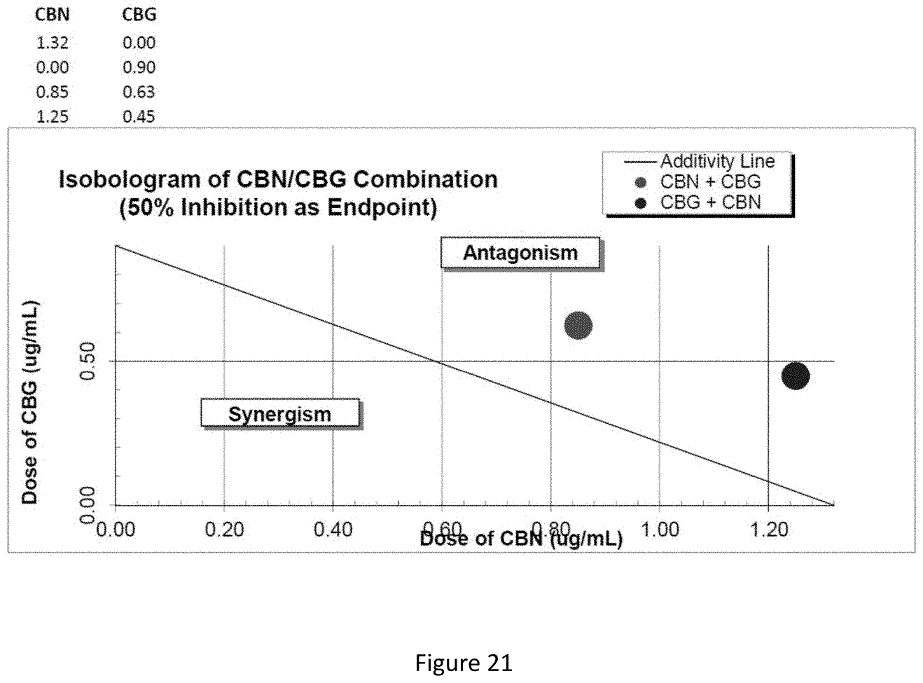

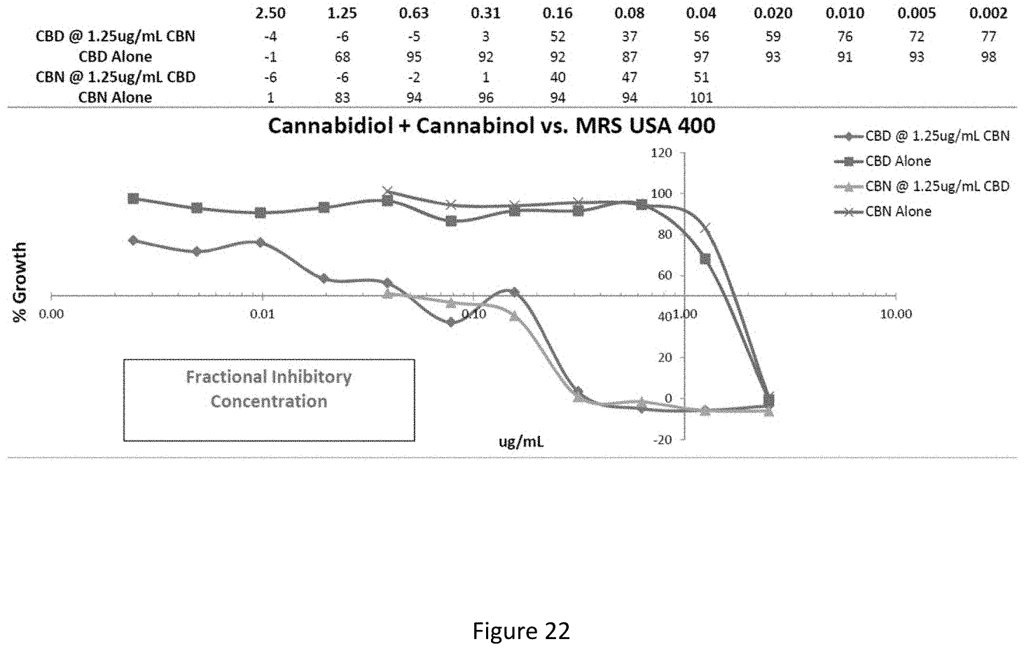

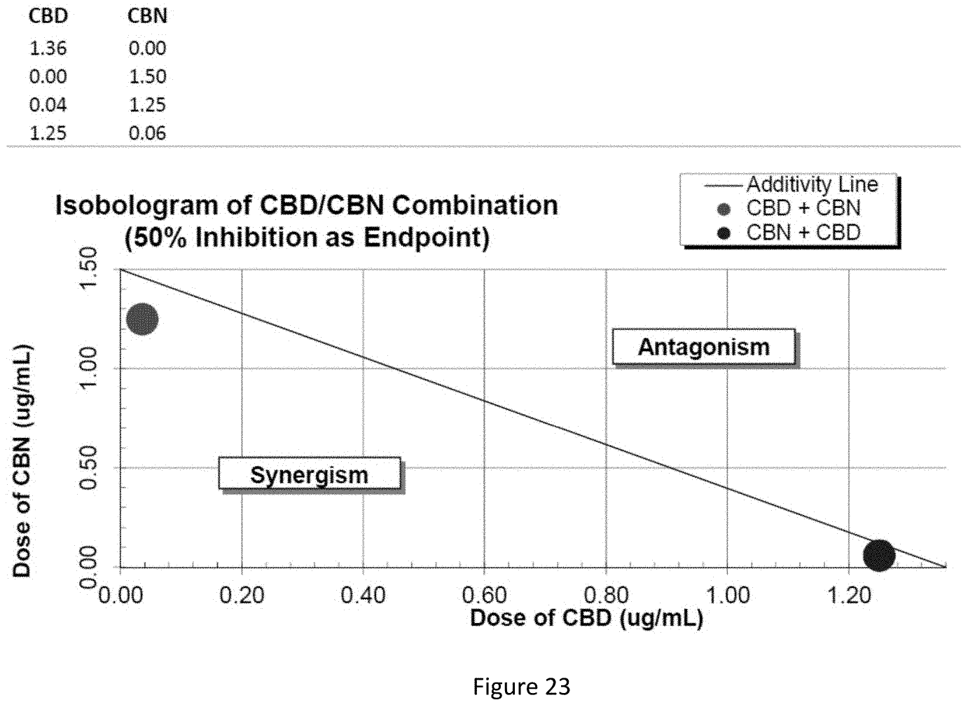

Table 12 demonstrates the IC₅₀s chosen for FICs against MRS 1696, A summary of the analysis of combinations of cannabinoids against MRS 1696 is presented in FIGS. 18-23.

TABLE 12
MRS 1696: IC_{50S} chosen for FICs

| Cannabidiol (CBD) + | | Cannabinol (CBN) + | | Cannabidiol (CBD) + | |
|---|---|---|---|---|---|
| Cannabigerol (CBG) | IC50 | Cannabigerol (CBG) | IC50 | Cannabinol (CBN) | IC50 |
| CBD @ 2.5 ug/mL CBG | <0.002 | CBN @ 2.5 ug/mL CBG | <0.002 | CBD @ 2.5 ug/mL CBN | <0.002 |
| CBD @ 1.25 ug/mL CBG | <0.002 | CBN @ 1.25 ug/mL CBG | <0.002 | CBD @ 1.25 ug/mL CBN | 0.036 |
| CBD @ 0.63 ug/mL CBG | 0.109 | CBN @ 0.63 ug/mL CBG | 0.854 | CBD @ 0.63 ug/mL CBN | 0.669 |
| CBD @ 0.31 ug/mL CBG | 0.666 | CBN @ 0.31 ug/mL CBG | 1.431 | CBD @ 0.31 ug/mL CBN | 0.759 |
| CBD @ 0.16 ug/mL CBG | 0.695 | CBN @ 0.16 ug/mL CBG | 1.996 | CBD @ 0.16 ug/mL CBN | 1.059 |
| CBD @ 0.08 ug/mL CBG | 0.912 | CBN @ 0.08 ug/mL CBG | 1.870 | CBD @ 0.08 ug/mL CBN | 1.186 |
| CBD @ 0.04 ug/mL CBG | 1.068 | CBN @ 0.04 ug/mL CBG | 1.384 | CBD @ 0.04 ug/mL CBN | 1.250 |
| CBD @ 0 ug/mL CBG | 1.277 | CBN @ 0 ug/mL CBG | 1.316 | CBD @ 0 ug/mL CBN | 1.363 |
| CBG @ 2.5 ug/mL CBD | <0.039 | CBG @ 2.5 ug/mL CBN | <0.039 | CBN @ 2.5 ug/mL CBD | <0.039 |
| CBG @ 1.25 ug/mL CBD | <0.039 | CBG @ 1.25 ug/mL CBN | 0.448 | CBN @ 1.25 ug/mL CBD | 0.058 |
| CBG @ 0.63 ug/mL CBD | 0.333 | CBG @ 0.63 ug/mL CBN | 0.786 | CBN @ 0.63 ug/mL CBD | 0.674 |
| CBG @ 0.31 ug/mL CBD | 0.453 | CBG @ 0.31 ug/mL CBN | 0.711 | CBN @ 0.31 ug/mL CBD | 0.821 |
| CBG @ 0.16 ug/mL CBD | 0.660 | CBG @ 0.16 ug/mL CBN | 0.799 | CBN @ 0.16 ug/mL CBD | 1.243 |
| CBG @ 0.08 ug/mL CBD | 0.648 | CBG @ 0.08 ug/mL CBN | 0.786 | CBN @ 0.08 ug/mL CBD | 1.116 |
| CBG @ 0.04 ug/mL CBD | 0.670 | CBG @ 0.04 ug/mL CBN | 0.988 | CBN @ 0.04 ug/mL CBD | 1.302 |
| CBG @ 0.02 ug/mL CBD | 0.675 | CBG @ 0.02 ug/mL CBN | 0.726 | CBN @ 0.02 ug/mL CBD | 1.274 |
| CBG @ 0.01 ug/mL CBD | 0.834 | CBG @ 0.01 ug/mL CBN | 1.101 | CBN @ 0.01 ug/mL CBD | 1.501 |
| CBG @ 0.005 ug/mL CBD | 0.729 | CBG @ 0.005 ug/mL CBN | 0.934 | CBN @ 0.005 ug/mL CBD | 1.449 |
| CBG @ 0.002 ug/mL CBD | 0.699 | CBG @ 0.002 ug/mL CBN | 0.826 | CBN @ 0.002 ug/mL CBD | 1.347 |
| CBG @ 0 ug/mL CBD | 0.900 | CBG @ 0 ug/mL CBN | 0.896 | CBN @ 0 ug/mL CBD | 1.499 |

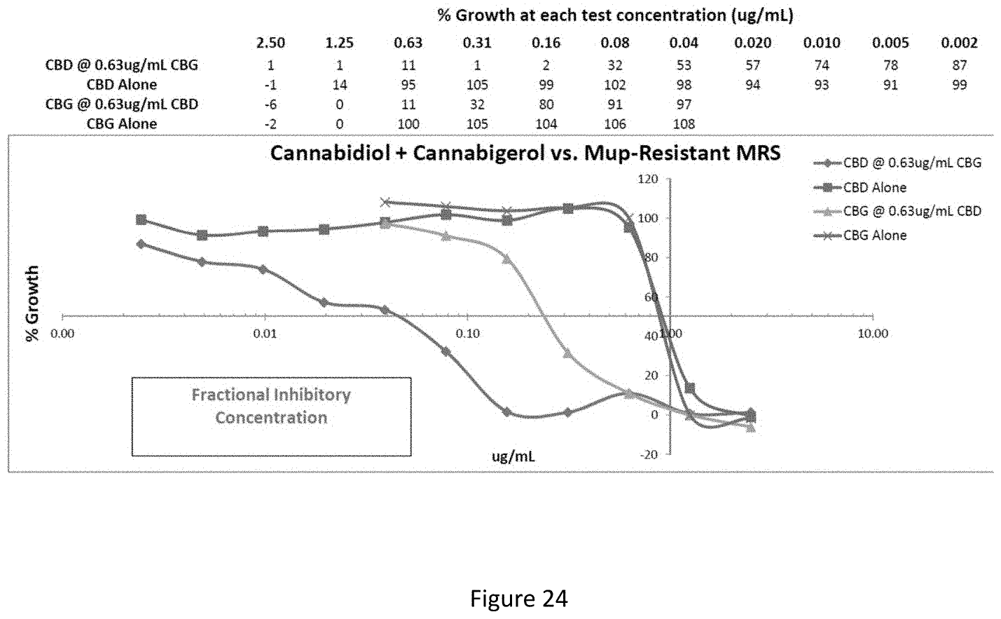

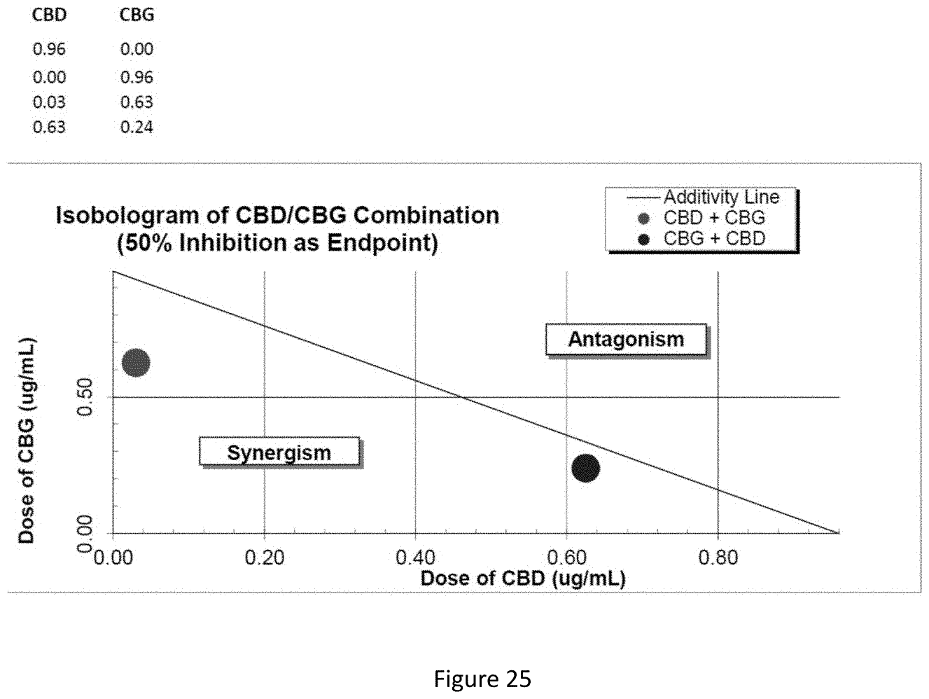

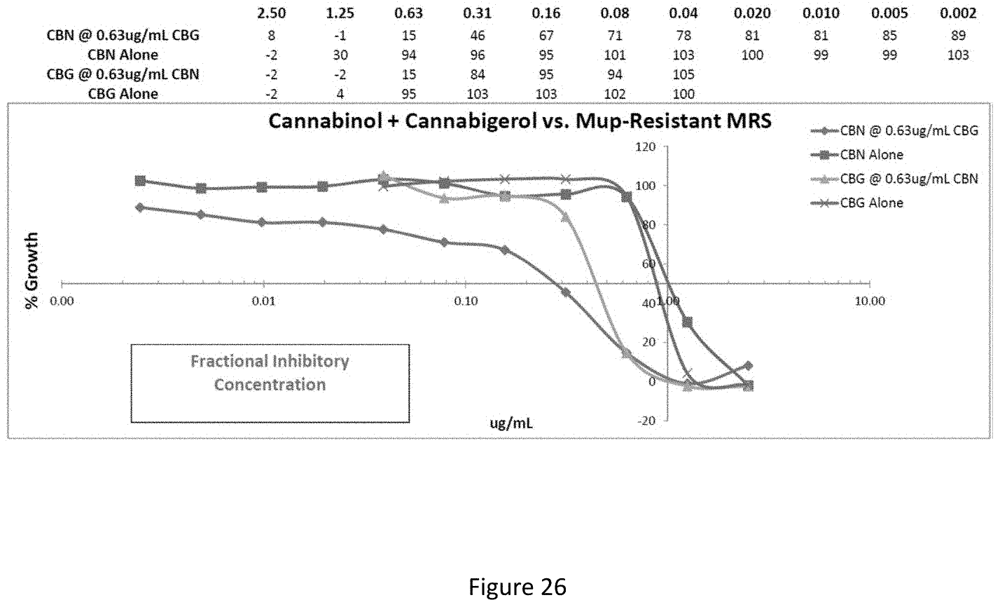

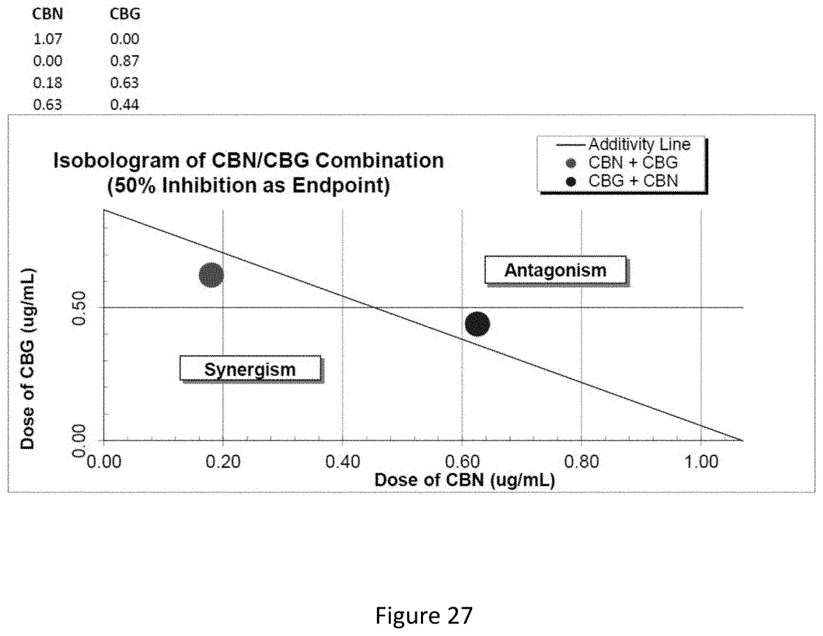

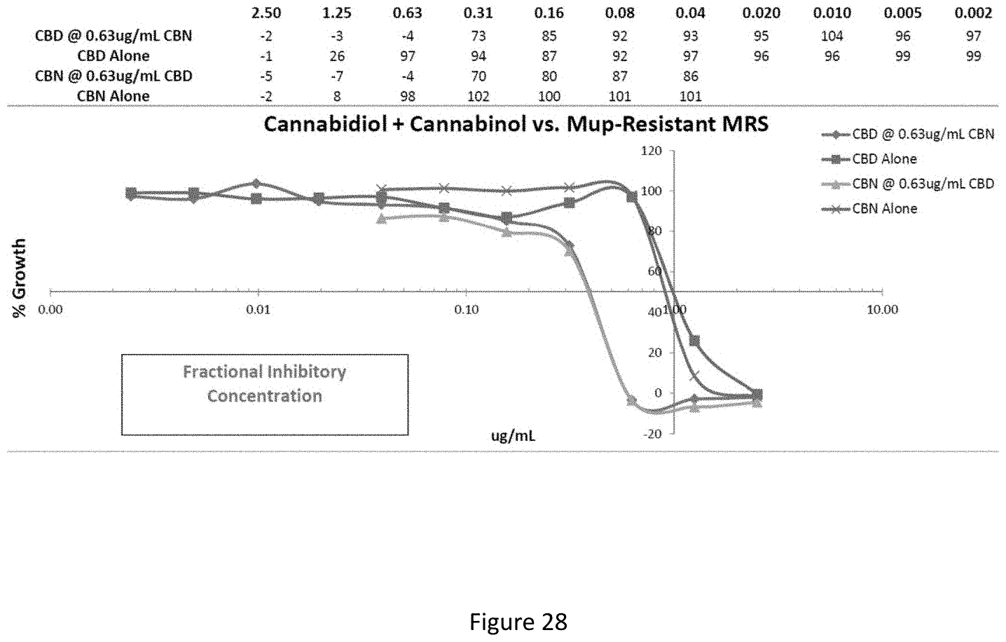

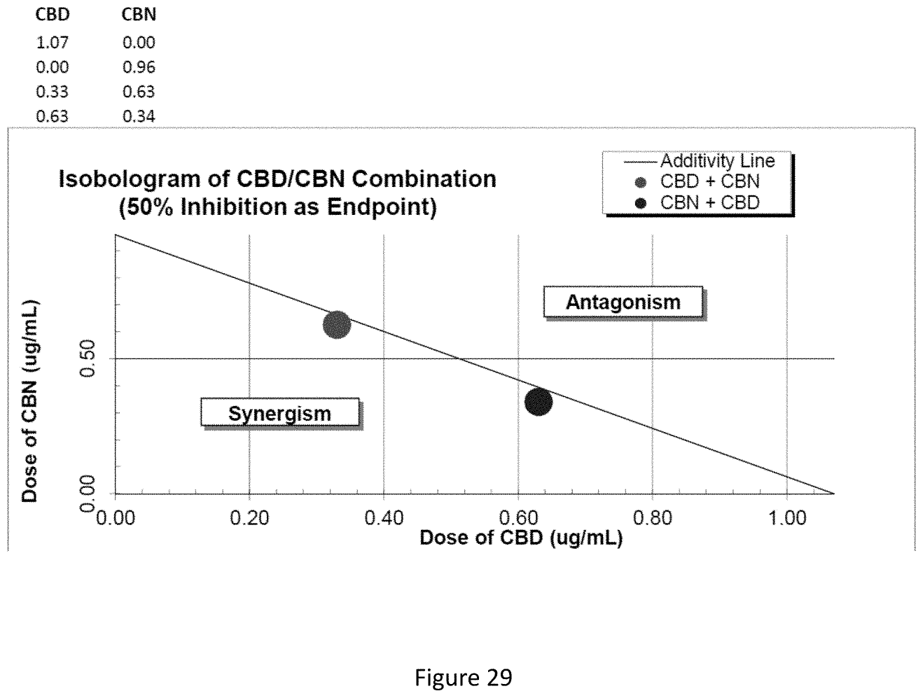

Table 13 demonstrates the IC₅₀s chosen for FICs against MRS 1708. A summary of the analysis of combinations of cannabinoids against MRS 1780 is presented in FIGS. 24-29.

TABLE 13
MRS 1708: IC_{50S} chosen for FICs

| Cannabidiol (CBD) + | | Cannabinol (CBN) + | | Cannabidiol (CBD) + | |
|---|---|---|---|---|---|
| Cannabigerol (CBG) | IC50 | Cannabigerol (CBG) | IC50 | Cannabinol (CBN) | IC50 |
| CBD @ 2.5 ug/mL CBG | <0.002 | CBN @ 2.5 ug/mL CBG | <0.002 | CBD @ 2.5 ug/mL CBN | <0.002 |
| CBD @ 1.25 ug/mL CBG | <0.002 | CBN @ 1.25 ug/mL CBG | <0.002 | CBD @ 1.25 ug/mL CBN | <0.002 |
| CBD @ 0.63 ug/mL CBG | 0.028 | CBN @ 0.63 ug/mL CBG | 0.177 | CBD @ 0.63 ug/mL CBN | 0.333 |
| CBD @ 0.31 ug/mL CBG | 0.440 | CBN @ 0.31 ug/mL CBG | 0.810 | CBD @ 0.31 ug/mL CBN | 0.659 |
| CBD @ 0.16 ug/mL CBG | 0.685 | CBN @ 0.16 ug/mL CBG | 0.983 | CBD @ 0.16 ug/mL CBN | 0.678 |
| CBD @ 0.08 ug/mL CBG | 0.725 | CBN @ 0.08 ug/mL CBG | 1.144 | CBD @ 0.08 ug/mL CBN | 0.702 |
| CBD @ 0.04 ug/mL CBG | 0.854 | CBN @ 0.04 ug/mL CBG | 1.242 | CBD @ 0.04 ug/mL CBN | 0.789 |
| CBD @ 0 ug/mL CBG | 0.960 | CBN @ 0 ug/mL CBG | 1.069 | CBD @ 0 ug/mL CBN | 1.065 |
| CBG @ 2.5 ug/mL CBD | <0.039 | CBG @ 2.5 ug/mL CBN | <0.039 | CBN @ 2.5 ug/mL CBD | <0.039 |
| CBG @ 1.25 ug/mL CBD | <0.039 | CBG @ 1.25 ug/mL CBN | <0.039 | CBN @ 1.25 ug/mL CBD | <0.039 |
| CBG @ 0.63 ug/mL CBD | 0.243 | CBG @ 0.63 ug/mL CBN | 0.437 | CBN @ 0.63 ug/mL CBD | 0.339 |
| CBG @ 0.31 ug/mL CBD | 0.355 | CBG @ 0.31 ug/mL CBN | 0.597 | CBN @ 0.31 ug/mL CBD | 0.707 |
| CBG @ 0.16 ug/mL CBD | 0.399 | CBG @ 0.16 ug/mL CBN | 0.655 | CBN @ 0.16 ug/mL CBD | 0.696 |
| CBG @ 0.08 ug/mL CBD | 0.533 | CBG @ 0.08 ug/mL CBN | 0.659 | CBN @ 0.08 ug/mL CBD | 0.716 |
| CBG @ 0.04 ug/mL CBD | 0.635 | CBG @ 0.04 ug/mL CBN | 0.66 | CBN @ 0.04 ug/mL CBD | 0.846 |
| CBG @ 0.02 ug/mL CBD | 0.650 | CBG @ 0.02 ug/mL CBN | 0.672 | CBN @ 0.02 ug/mL CBD | 0.724 |
| CBG @ 0.01 ug/mL CBD | 0.664 | CBG @ 0.01 ug/mL CBN | 0.678 | CBN @ 0.01 ug/mL CBD | 0.733 |
| CBG @ 0.005 ug/mL CBD | 0.670 | CBG @ 0.005 ug/mL CBN | 0.684 | CBN @ 0.005 ug/mL CBD | 0.729 |
| CBG @ 0.002 ug/mL CBD | 0.700 | CBG @ 0.002 ug/mL CBN | 0.701 | CBN @ 0.002 ug/mL CBD | 0.918 |
| CBG @ 0 ug/mL CBD | 0.959 | CBG @ 0 ug/mL CBN | 0.872 | CBN @ 0 ug/mL CBD | 0.956 |

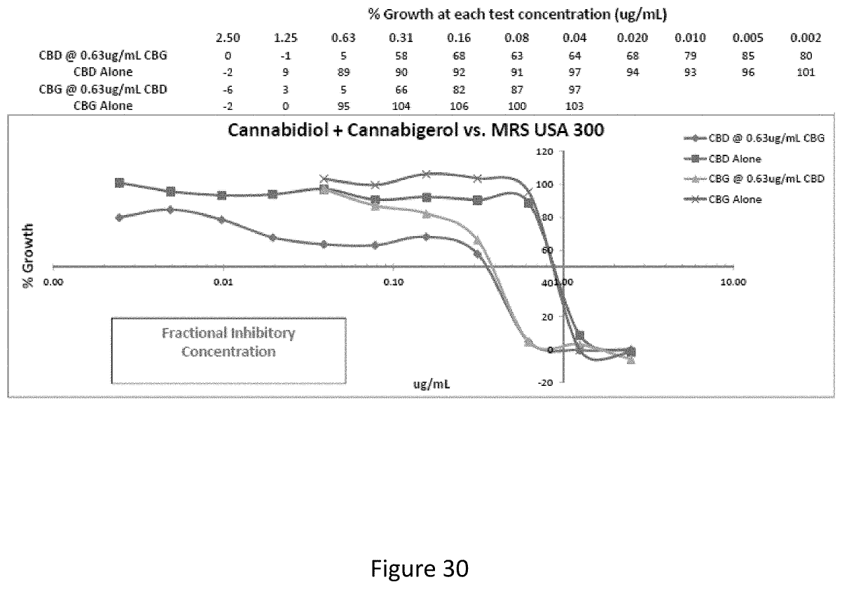

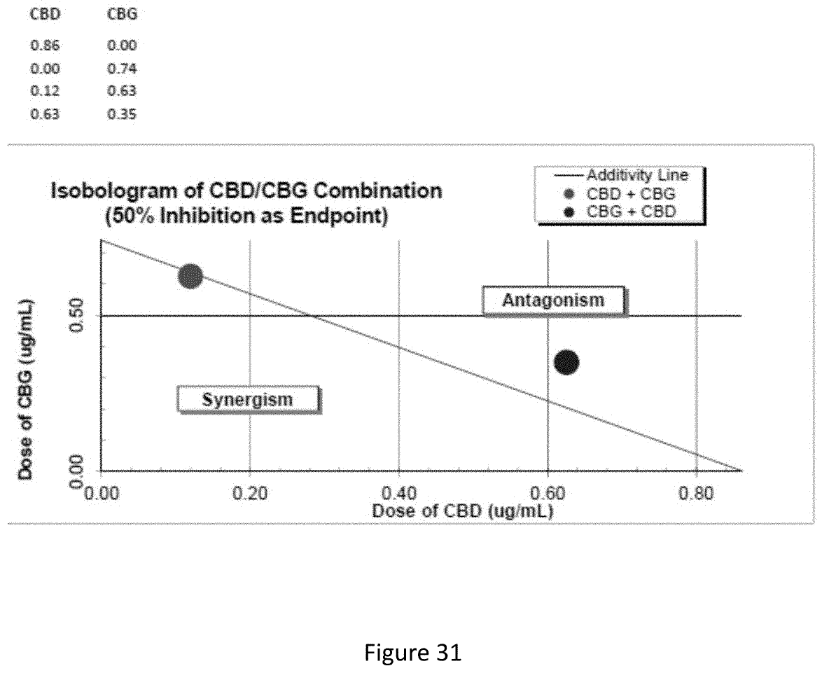

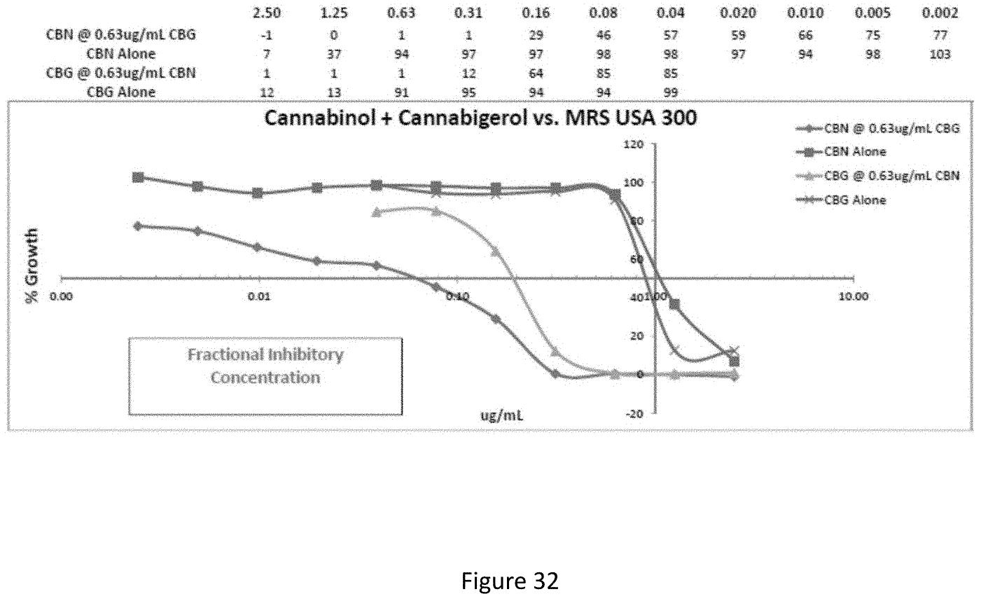

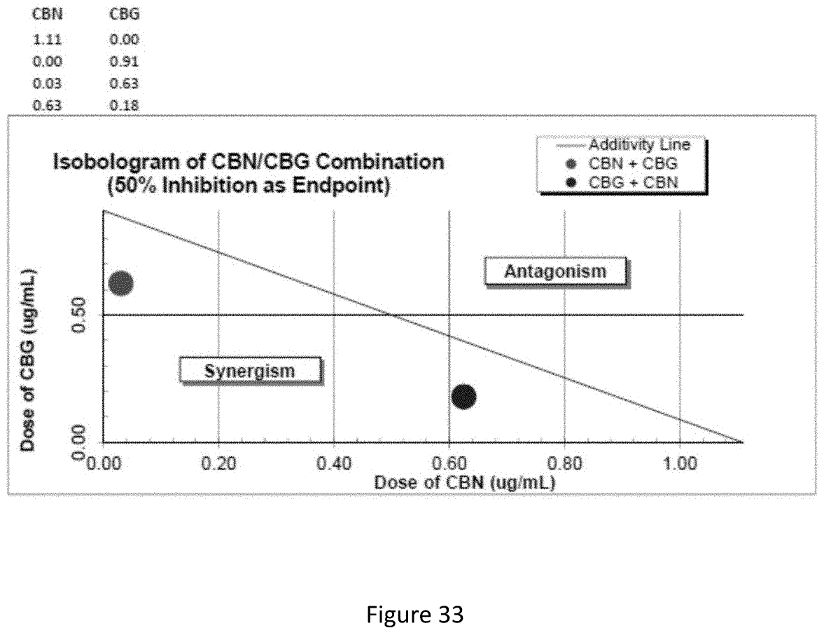

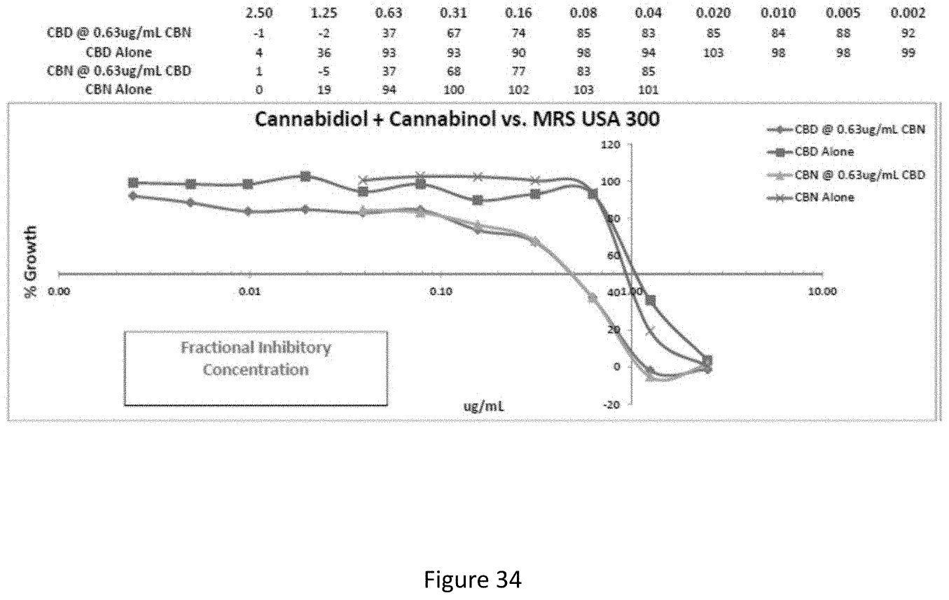

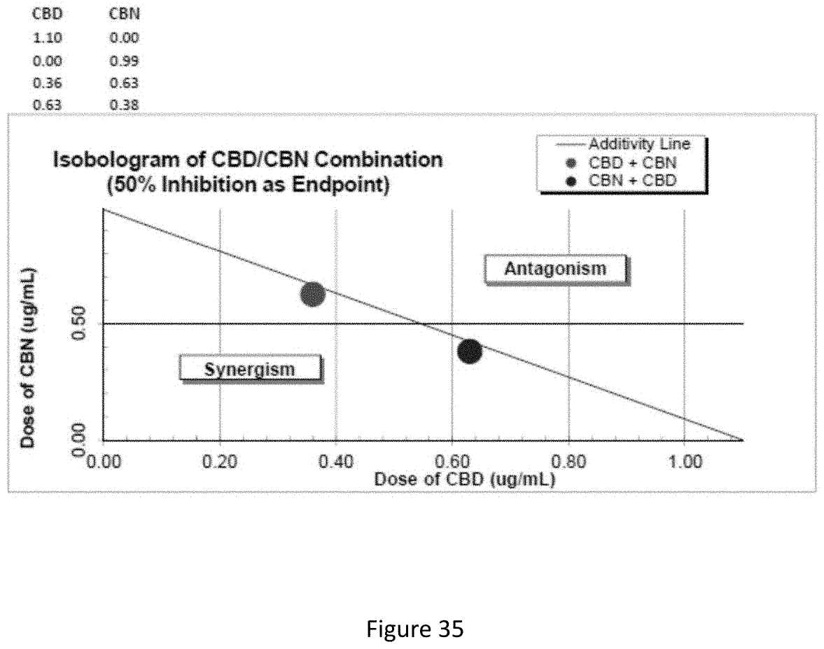

Table 14 demonstrates the IC₅₀s chosen for FICs against MRS 1717. A summary of the analysis of combinations of cannabinoids against MRS 1717 is presented in FIGS. 30-35.

TABLE 14
MRS 1717: IC_{50S} chosen for FICs

| Cannabidiol (CBD) + | | Cannabinol (CBN) + | | Cannabidiol (CBD) + | |
|---|---|---|---|---|---|
| Cannabigerol (CBG) | IC50 | Cannabigerol (CBG) | IC50 | Cannabinol (CBN) | IC50 |
| CBD @ 2.5 ug/mL CBG | <0.002 | CBN @ 2.5 ug/mL CBG | <0.002 | CBD @ 2.5 ug/mL CBN | <0.002 |
| CBD @ 1.25 ug/mL CBG | <0.002 | CBN @ 1.25 ug/mL CBG | <0.002 | CBD @ 1.25 ug/mL CBN | <0.002 |
| CBD @ 0.63 ug/mL CBG | 0.120 | CBN @ 0.63 ug/mL CBG | 0.031 | CBD @ 0.63 ug/mL CBN | 0.356 |
| CBD @ 0.31 ug/mL CBG | 0.706 | CBN @ 0.31 ug/mL CBG | 0.356 | CBD @ 0.31 ug/mL CBN | 0.679 |
| CBD @ 0.16 ug/mL CBG | 0.754 | CBN @ 0.16 ug/mL CBG | 0.694 | CBD @ 0.16 ug/mL CBN | 0.680 |
| CBD @ 0.08 ug/mL CBG | 0.784 | CBN @ 0.08 ug/mL CBG | 0.789 | CBD @ 0.08 ug/mL CBN | 0.828 |
| CBD @ 0.04 ug/mL CBG | 0.910 | CBN @ 0.04 ug/mL CBG | 0.755 | CBD @ 0.04 ug/mL CBN | 0.901 |
| CBD @ 0 ug/mL CBG | 0.864 | CBN @ 0 ug/mL CBG | 1.108 | CBD @ 0 ug/mL CBN | 1.098 |
| CBG @ 2.5 ug/mL CBD | <0.039 | CBG @ 2.5 ug/mL CBN | <0.039 | CBN @ 2.5 ug/mL CBD | <0.039 |
| CBG @ 1.25 ug/mL CBD | <0.039 | CBG @ 1.25 ug/mL CBN | <0.039 | CBN @ 1.25 ug/mL CBD | <0.039 |
| CBG @ 0.63 ug/mL CBD | 0.315 | CBG @ 0.63 ug/mL CBN | 0.176 | CBN @ 0.63 ug/mL CBD | 0.383 |
| CBG @ 0.31 ug/mL CBD | 0.651 | CBG @ 0.31 ug/mL CBN | 0.349 | CBN @ 0.31 ug/mL CBD | 0.694 |
| CBG @ 0.16 ug/mL CBD | 0.725 | CBG @ 0.16 ug/mL CBN | 0.484 | CBN @ 0.16 ug/mL CBD | 0.683 |
| CBG @ 0.08 ug/mL CBD | 0.682 | CBG @ 0.08 ug/mL CBN | 0.579 | CBN @ 0.08 ug/mL CBD | 1.046 |
| CBG @ 0.04 ug/mL CBD | 0.687 | CBG @ 0.04 ug/mL CBN | 0.653 | CBN @ 0.04 ug/mL CBD | 0.863 |
| CBG @ 0.02 ug/mL CBD | 0.727 | CBG @ 0.02 ug/mL CBN | 0.667 | CBN @ 0.02 ug/mL CBD | 0.893 |
| CBG @ 0.01 ug/mL CBD | 0.813 | CBG @ 0.01 ug/mL CBN | 0.687 | CBN @ 0.01 ug/mL CBD | 0.729 |
| CBG @ 0.005 ug/mL CBD | 0.801 | CBG @ 0.005 ug/mL CBN | 0.733 | CBN @ 0.005 ug/mL CBD | 0.732 |
| CBG @ 0.002 ug/mL CBD | 0.766 | CBG @ 0.002 ug/mL CBN | 0.725 | CBN @ 0.002 ug/mL CBD | 0.780 |
| CBG @ 0 ug/mL CBD | 0.741 | CBG @ 0 ug/mL CBN | 0.911 | CBN @ 0 ug/mL CBD | 0.908 |

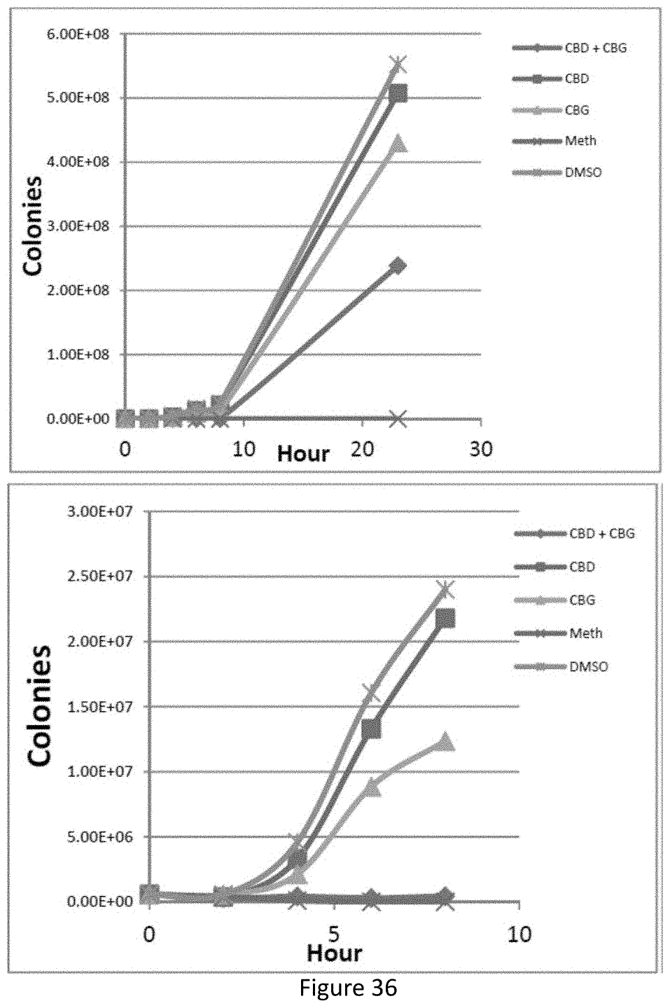

FIG. 36 shows the time kill of cannabidiol and cannabigerol vs, Mupirocin-resistant MRS. Organisms include Mupirocin-resistant S. aureus (ATCC BAA-1708). Strains were grown on Eugon agar at 35° C. 18-20 hours prior to assays. Test samples were dissolved in DMSO (2mg/mL). The combination of CBD+CBG: 144688 (Cannabidiol)+144675 (Cannabigerol)®0.5/0.5 ug/mL was tested against single CBD: 144688 (Cannabidiol) Alone at 0.5 ug/mL; single CBG: 144675 (Cannabigerol) alone at 0.5 ug/mL; Methicillin at 50 ug/mL; and DMSO. At various timepoints, samples were removed and diluted, transferred to agar and incubated at 35° C. Colonies were counted after 24 hours incubation.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising two or more cannabinoids.

2. The composition of claim 1, wherein the two or more cannabinoids are selected from the group consisting of: Cannabidiol (CBD), a CBD derivative, Cannabinol (CBN), a CBN derivative, Cannabigerol (CBG), and a CBG derivative.

3. The composition of claim 2, wherein the composition comprises CBD and CBN.

4. The composition of claim 2, wherein the composition comprises CBD and CBG.

5. The composition of claim 2, wherein the composition comprises CBG and CBN.

6. The composition of claim 2, wherein the composition comprises CBD, CBG and CBN.

7. The composition of claim 2, wherein the CBD derivative is selected from the group consisting of Cannabidiolic acid, Monomethoxy cannabidiol, Cannabielsoin, Cannabichromene, and Cannabichromeorcin.

8. The composition of claim 2, wherein the CBN derivative is selected from. the group consisting of 8-Hydroxycannabinol, Cannabinolic acid, and Cannabinol acetate.

9. The composition of claim 2, wherein the CBG derivative is selected from the group consisting of Tetrahydrocannabigerol, 6,7-Epoxycannabigerol, Cannabigerolic acid, Tetrahydrocannabigerolic acid, (Z)-Cannabigerorcin, and Methyl tetrahydrocannabigerol.

10. The composition of claim 1, wherein the composition further comprises an additional therapeutic.

11. The composition of claim 10, wherein the additional therapeutic is an antibiotic, Tetrahydrocannabinol (Δ8-THC) or a Δ8-THC derivative.

12. A pharmaceutical composition comprising the composition of claim 1.

13. The pharmaceutical composition of claim 12, wherein the pharmaceutical composition is selected from the group consisting of a topical ointment, a topical cream, a topical gel, a topical lotion, a topical paste, a topically applied patch, a microparticle, suppository, and a nasal spray.

14. A method of treating a bacterial infection or a disease or disorder associated with a bacterial infection in a subject, the method comprising administering an effective amount of two or more cannabinoids.

15. The method of claim 14, wherein the two or more cannabinoids are selected from the group consisting of: Cannabidiol (CBD), a CBD derivative, Cannabinol (CBN), a CBN derivative, Cannabigerol (CBG), and a CBG derivative.

16. The method of claim 14, wherein the bacterial infection is a gram-positive bacterial infection.

17. The method of claim 16, wherein the gram-positive bacterial infection is a bacterial infection of Staphylococcus aureus, Hemolytic Stept, P. acnes, Clostridium, Bacillus, or Listeria.

18. The method of claim 14, wherein the disease or disorder associated with a bacterial infection is selected from the group consisting of cellulitis, impetigo, erysipelas, boils, acne, abscesses, epidermolysis bullosa, a bullous disease, toxic epidermal necrolysis, an infection secondary to environmental allergens (or drug reactions, and staphylococcal scalded skin syndrome.

19. The method of claim 14, wherein the two or more cannabinoids are administered concurrently.

20. A method of preventing or reducing the growth or proliferation of microorganisms and/or biofilm-embedded microorganisms on at least one surface, the method comprising providing at least one surface; providing a composition comprising at least two cannabinoids, and applying the composition to the at least one surface in an amount sufficient to prevent or reduce the growth or proliferation of microorganisms or biofilm-embedded microorganisms on the at least one surface.

21. The method of claim 20, wherein the two or more cannabinoids are selected from the group consisting of: Cannabidiol (CBD), a CBD derivative, Cannabinol (CBN), a CBN derivative, Cannabigerol (CBG), and a CBG derivative.

22. The method of claim 20, wherein the surface is selected from the group consisting of a subject's body, a medical device, and a solid surface.
